SEQUENCE LISTING (2 sequences)

SEQ ID NO: 1 - DNA, 17 bp, synthetic construct
  source 1..17: mol_type = other DNA
ctcctggaaacgggtgg

SEQ ID NO: 2 - DNA, 22 bp, synthetic construct
  source 1..22: mol_type = other DNA
ggtgttcttcccgatatctaca

(19) United States
(12) Patent Application Publication
(10) Pub. No.: US 2025/0120941 A1
(43) Pub. Date: Apr. 17, 2025

(54) Composition for Promoting the Assimilation of an Oligosaccharide

(71) Applicant: MORINAGA MILK INDUSTRY CO., LTD., Minato-ku, Tokyo (JP)

(72) Inventors: Jier Xi, Zama-shi, Kanagawa (JP); Tatsuya Ehara, Zama-shi, Kanagawa (JP); Takuya Kamimura, Zama-shi, Kanagawa (JP); Muneya Tsuda, Zama-shi, Kanagawa (JP)

(73) Assignee: MORINAGA MILK INDUSTRY CO., LTD., Minato-ku, Tokyo (JP)

(21) Appl. No.: 18/702,300

(22) PCT Filed: Oct. 21, 2022
(86) PCT No.: PCT/JP2022/039413

§ 371 Date: Apr. 17, 2024

(30) Foreign Application Priority Data
Oct. 21, 2021 (JP) 2021-172612
Oct. 21, 2021 (JP) 2021-172613

(51) Int. Cl. A61K 31/355; A61K 31/702; A61K 35/00; A61K 35/20; A61K 35/745; A61P 1/14

(52) U.S. Cl. CPC A61K 31/355; A61K 31/702; A61K 35/20; A61K 35/745; A61P 1/14; A61K 2035/115

(57) ABSTRACT

A technique for promoting the assimilation of an oligosaccharide such as an HMO by an intestinal bacterium, especially a bifidobacterial strain, is provided. As a first aspect, a composition for promoting the assimilation of an oligosaccharide containing a tocopherol is provided. As a second aspect, a nutritional composition (excluding breast milk) containing an oligosaccharide and a tocopherol is provided. The tocopherol in the aspects contains one, two or more kinds selected from the group consisting of β-tocopherol, γ-tocopherol and δ-tocopherol. The oligosaccharide in the aspects is preferably a human milk oligosaccharides. The mass ratio of the δ-tocopherol content to the β-tocopherol content (δ/β) in the aspects is preferably 5 or more, and the mass ratio of the γ-tocopherol content to the δ-tocopherol content (γ/δ) is preferably 8.8 or less.

### TECHNICAL FIELD

The present invention relates to new use of a tocopherol and specifically relates to use for promoting the assimilation of an oligosaccharide. The invention also relates to a nutritional composition containing an oligosaccharide and a tocopherol.

### BACKGROUND ART

In recent years, various studies on human milk oligosaccharides (also referred to as HMOs below), a generic term for many kinds of oligosaccharide contained in human colostrum, have been made. It is calculated that human milk oligosaccharides are contained at 22 to 24 g/L in colostrum and at 12 to 13 g/L in normal milk, and human milk oligosaccharides are the third highest solid content in breast milk after lactose and lipids. Human milk oligosaccharides are known to have an infection-protecting action, an immunomodulatory action and the like.

It is believed that oligosaccharides including human milk oligosaccharides are assimilated by various bacteria in the intestines and thus contribute to the health of the host (NPL 1). In particular, applications of oligosaccharides themselves including HMOs and sugar sources which are combinations of oligosaccharides and other components as “prebiotics” which are assimilated by intestinal bacteria beneficial for human and which proliferate the bacteria have been proposed and studied intensively.

For example, PTL 1 discloses that HMOs are selectively utilized by bifidobacterial strains in the intestines of infants and promote the growth thereof. Of intestinal bacteria, bifidobacterial strains are known to be the most dominant in the enteric microbiota of infants and young children. bifidobacterial strains are obligate anaerobes living in the lower part of the small intestine to the large intestine of healthy humans and are widely known to play an important role in maintaining the health of the host due to the intestinal regulation action, the immunomodulatory action and the like (NPL 2).

Accordingly, for enhancing the health of humans including infants and young children, it is important to develop a composition which improves the easy assimilation of oligosaccharides such as HMOs in the intestines, particularly to develop a composition which is easily assimilated by a bifidobacterial strain and which promotes the growth thereof.

Tocopherols have attracted attention as components that are blended in a nutritional composition for infants and young children, and it has been reported that α-tocopherol promotes the growth of bifidobacterial strains (PTL 2).

PTL 3 discloses a composition containing 2′-fucosyllactose, which is one of HMOs, and α-tocopherol and describes that the composition has an action of improving at least one of cognition, learning and memory.

In this regard, other isoforms of tocopherol than α-tocopherol are still under study, and sufficient findings have not been made.

### CITATION LIST

### Patent Literature

PTL 1: WO2012-158517
PTL 2: WO2018-237149
PTL 3: WO2016-086151

### Non-Patent Literature

NPL 1: L. W. Chia et al., Microorganisms, 2020, 8 (10), 1513
NPL 2: T. Urashima et al., Milk Science Vol. 56, No. 4, 2008

### SUMMARY OF INVENTION

### Technical Problem

A problem of the invention is to provide a technique for promoting the assimilation of an oligosaccharide such as an HMO by an intestinal bacterium, especially a bifidobacterial strain.

### Solution to Problem

As a result of intensive study to solve the problem, the present inventors have found that an isoform of tocopherol other than α-tocopherol, namely one, two or more kinds selected from the group consisting of β-tocopherol, γ-tocopherol and δ-tocopherol, promotes the assimilation of an oligosaccharide such as a human milk oligosaccharides by an intestinal bacterium. The inventors have also found that, when an oligosaccharide such as a human milk oligosaccharides and an isoform of tocopherol other than α-tocopherol are combined, the growth of a bifidobacterial strain can be further promoted, and the invention has been thus completed.

That is, a first aspect of the invention is a composition comprising a tocopherol for promoting the assimilation of an oligosaccharide, wherein the tocopherol contains one, two or more kinds selected from the group consisting of β-tocopherol, γ-tocopherol and δ-tocopherol. The terms “assimilation of an oligosaccharide” and “utilization of an oligosaccharide” can interchangeably be used herein. “Assimilation of an oligosaccharide” as used herein refers to the consumption of an oligosaccharide. The consumption of an oligosaccharide can readily be determined by measuring the change of the level/amount of an oligosaccharide in a medium during the cultivation over time (e.g. by measuring the level/amount at the start of the cultivation and at later point(s) of time, e.g. after 7, 10 or 24 hours). The term “assimilation of an oligosaccharide” can include or be “metabolization of an oligosaccharide”. It is understood that these terms refer primarily to assimilation or utilization or metabolization or consumption, respectively, by intestinal bacteria, particularly bifidobacteria.

Further, the terms “comprising” and “containing” are used interchangeably herein.

The explanations and definitions given in relation to the composition of the first aspect (particularly the “nutritional composition”) apply, mutatis mutandis, to the composition of the second aspect (particularly the “nutritional composition”) and vice versa.

In the composition of the aspect, the mass ratio of the δ-tocopherol content to the β-tocopherol content (δ/β) is preferably 5 or more, more preferably 10 or more. In the composition of the aspect, the mass ratio of the γ-tocopherol content to the δ-tocopherol content (γ/δ) is preferably 8.8 or less, more preferably 3.9 or less.

More preferable use of the composition of the aspect is use for promoting the assimilation of an oligosaccharide having three or more and five or less sugar residues. More preferable use of the composition of the aspect is use for promoting the assimilation of a human milk oligosaccharides. The human milk oligosaccharides whose assimilation is promoted in the use preferably contains one, two or more kinds selected from the group consisting of lacto-N-neotetraose (also referred to as LNnT), 2′-fucosyllactose (also referred to as 2′-FL), 3′-sialyllactose (also referred to as 3′-SL), 6′-sialyllactose (also referred to as 6′-SL), difucosyllactose (DFL) and 3-fucosyllactose (3-FL). The composition of the aspect may further contain a human milk oligosaccharides. In the aspect, the composition preferably contains lacto-N-tetraose (also referred to as LNT) and is preferably for use for promoting the assimilation of a human milk oligosaccharides containing 2′-fucosyllactose.

The composition of the aspect is preferably a nutritional composition, more preferably formula, particularly preferably a milk formula. The nutritional composition preferably is an infant formula, follow-up formula, or a growing-up formula. An “infant formula” as used herein refers to a formula intended for use by an infant aged 0 to 6 months (i.e. during the first 6 months of life). A “follow up formula” as used herein refers to a formula intended for use by an infant aged 6 to 12 months (i.e. 6 months of age and older, up to and including 12 months). The terms follow-up formula and follow-on formula are used interchangeably herein. A “growing-up formula” as used herein refers to a formula intended for use by a child aged 12 to 36 months (i.e. a child at the age of 36 months or under the age of 36 months and older than 12 months). The terms “growing-up formula” and “formula for a young child/young children” are used interchangeably herein.

The term “infant” means a child which is 0 to 12 months old (in other words a child at the age of 12 months or under the age of 12 months). The term “young child” means a child which is between 12 to 36 months old (in other words a child at the age of 36 months or under the age of 36 months and older than 12 months). A “young child” is also referred to herein as “toddler”.

The composition of the aspect may be an aspect which is added to a composition containing a human milk oligosaccharides before use. In the aspect, the composition containing the human milk oligosaccharides is preferably breast milk, particularly human breast milk, such as pumped breast milk. The terms “pumped breast milk” and “expressed breast milk” are used interchangeably herein. Thus, it is envisaged herein that the composition of the invention (particularly the nutritional composition) can be used as an additive or supplement, which preferably can be added to another composition containing a human milk oligosaccharide, such as breast milk. Thereby the nutritional value of the other composition (such as breast milk) can be enhanced. It is envisaged that the composition containing a human milk oligosaccharide, such as breast milk, to which the composition of the invention was added, (i.e. the prepared mixture) is taken by an infant/child or is fed to the infant/child.

In accordance with the above, the invention relates in one aspect to the use of a composition comprising a tocopherol for promoting the assimilation of an oligosaccharide,
wherein the tocopherol contains one, two or more kinds selected from the group consisting of β-tocopherol, γ-tocopherol and δ-tocopherol.

The invention relates in one aspect, to the use of a composition disclosed herein, specifically a nutritional composition, for promoting the growth of a bifidobacterial strain. In this context of “promoting the assimilation of an oligosaccharide” or “for promoting the growth of a bifidobacterial strain”, the composition can be used e.g. as an additive or supplement which preferably can be added to another composition containing a human milk oligosaccharide, such as breast milk.

In one aspect, the use or method disclosed herein, particularly in this context, is an in vitro or ex vivo use.

In one aspect, the use or method for gut flora improvement disclosed herein is non-therapeutic.

In one aspect, the method for gut flora improvement disclosed herein is not a method for treatment of the human or animal body by therapy. In one aspect, the use for gut flora improvement disclosed herein is not a method for treatment of the human or animal body by therapy.

A second aspect of the invention is a nutritional composition (excluding breast milk) comprising an oligosaccharide and a tocopherol in which the tocopherol contains one, two or more kinds selected from the group consisting of β-tocopherol, γ-tocopherol and δ-tocopherol.

In the nutritional composition of the aspect, the mass ratio of the δ-tocopherol content to the β-tocopherol content (δ/β) is preferably 5 or more, more preferably 10 or more.

In the nutritional composition of the aspect, the mass ratio of the γ-tocopherol content to the δ-tocopherol content (γ/δ) is preferably 8.8 or less, more preferably 3.9 or less.

In the nutritional composition of the aspect, the mass ratio of the total amount of β-tocopherol, γ-tocopherol and δ-tocopherol to the oligosaccharide content is preferably 0.001 to 0.2.

In the nutritional composition of the aspect, the total amount of β-tocopherol, γ-tocopherol and δ-tocopherol based on the entire composition is preferably 0.0002 to 0.1 mass %.

In the nutritional composition of the aspect, the oligosaccharide content of the entire composition is preferably 0.05 to 10 mass %.

In the nutritional composition of the aspect, the oligosaccharide preferably has three or more and five or less sugar residues. The oligosaccharide is preferably a human milk oligosaccharides and more preferably contains one, two or more kinds selected from the group consisting of lacto-N-neotetraose, lacto-N-tetraose, 2′-fucosyllactose, 3′-sialyllactose, 6′-sialyllactose, difucosyllactose and 3-fucosyllactose. In particular, an embodiment containing lacto-N-tetraose and 2′-fucosyllactose as oligosaccharides is preferable. An embodiment containing fructooligosaccharide and 6′-sialyllactose is also preferable.

The nutritional composition of the aspect is suitable for use for promoting the growth of a bifidobacterial strain, and the bifidobacterial strain is preferably one, two or more strains selected from the group consisting of Bifidobacterium breve, Bifidobacterium bifidum, Bifidobacterium infantis and Bifidobacterium longum.

The nutritional composition of the aspect preferably further contains a bifidobacterial strain.

The nutritional composition of the aspect is preferably a formula.

### Advantageous Effects of Invention

According to the invention, a composition which can promote the assimilation of an oligosaccharide such as an HMO by an intestinal bacterium is provided. Moreover, according to the invention, a nutritional composition which can promote the growth of a bifidobacterial strain in the intestines is provided.

The composition can be an aspect as an oral composition such as a food, a drink or a pharmaceutical product or can be contained in a food, a drink or a pharmaceutical production as an aspect as an additive or the like. By taking the composition of the invention, it is expected that an effect of improving the enteric microbiota is obtained and that the intake helps maintaining the health of the person taking the composition, especially an infant or a young child.

### BRIEF DESCRIPTION OF DRAWINGS

FIG. 1 A graph showing the growth of Bifidobacterium infantis M-63 with the addition of tocopherols (n=6). The differences of OD₆₀₀ values between the start and the end of 16 hour culturing are shown.

FIG. 2 A graph showing the increase degrees of bacterial cells (the differences from the initial values at OD₆₀₀) after culturing Bifidobacterium infantis M-63 alone for 16 hours with the addition of tocopherols and 2′-fucosyllactose (n=6) The term “increase degrees of bacterial cells” as used herein refers to the “differences of OD₆₀₀ values between the start and the end of culturing”.

FIG. 3 A graph showing the increase degrees of bacterial cells (the differences from the initial values at OD₆₀₀) after culturing Bifidobacterium infantis M-63 alone for 16 hours with the addition of tocopherols and 6′-sialyllactose (n=6).

FIG. 4 A graph showing the increase degrees of bacterial cells (the differences from the initial values at OD₆₀₀) after culturing Bifidobacterium infantis M-63 alone for 16 hours with the addition of tocopherols and galactooligosaccharide (n=6).

FIG. 5 A graph showing the increase degrees of bacterial cells (the differences from the initial values at OD₆₀₀) after culturing Bifidobacterium infantis MCC2042 alone for 16 hours with the addition of tocopherols (n=6).

FIG. 6 A graph showing the increase degrees of bacterial cells (the differences from the initial values at OD₆₀₀) after culturing Bifidobacterium infantis MCC2042 alone for 16 hours with the addition of tocopherols and 2′-fucosyllactose (n=6).

FIG. 7 A graph showing the increase degrees of bacterial cells (the differences from the initial values at OD₆₀₀) after culturing Bifidobacterium infantis MCC2042 alone for 16 hours with the addition of tocopherols and 6′-sialyllactose (n=6).

FIG. 8 A graph showing the increase degrees of bacterial cells (the differences from the initial values at OD₆₀₀) after culturing Bifidobacterium infantis MCC2042 alone for 16 hours with the addition of tocopherols and galactooligosaccharide (n=6).

FIG. 9 A graph showing the increase degrees of bacterial cells (the differences from the initial values at OD₆₀₀) after culturing Bifidobacterium bifidum MCC2030 alone for 16 hours with the addition of tocopherols (n=6).

FIG. 10 A graph showing the increase degrees of bacterial cells (the differences from the initial values at OD₆₀₀) after culturing Bifidobacterium bifidum MCC2030 alone for 16 hours with the addition of tocopherols and 2′-fucosyllactose (n=6).

FIG. 11 A graph showing the increase degrees of bacterial cells (the differences from the initial values at OD₆₀₀) after culturing Bifidobacterium bifidum MCC2030 alone for 16 hours with the addition of tocopherols and 6′-sialyllactose (n=6).

FIG. 12 A graph showing the increase degrees of bacterial cells (the differences from the initial values at OD₆₀₀) after culturing Bifidobacterium bifidum MCC2030 alone for 16 hours with the addition of tocopherols and galactooligosaccharide (n=6).

### DESCRIPTION OF EMBODIMENTS

Next, the invention is explained in detail. In this regard, however, the invention is not limited to the following embodiments, and changes can be freely made in the scope of the invention.

In certain aspects, the invention also relates to the following items.

### Item 1:

Use of a composition comprising a tocopherol for promoting the assimilation of an oligosaccharide, wherein the tocopherol contains one, two or more kinds selected from the group consisting of β-tocopherol, γ-tocopherol and δ-tocopherol.

### Item 2:

Use of a composition for gut flora improvement in a subject, the composition comprising a tocopherol, wherein the tocopherol contains one, two or more kinds selected from the group consisting of β-tocopherol, γ-tocopherol and δ-tocopherol.

### Item 3:

A method for gut flora improvement wherein a composition comprising a tocopherol is administered to a subject, wherein the tocopherol contains one, two or more kinds selected from the group consisting of β-tocopherol, γ-tocopherol and δ-tocopherol.

### Item 4:

The use of item 1 or 2, or the method of item 3, wherein the subject is a healthy subject.

### Item 5:

A composition comprising a tocopherol for use in preventing or treating a disease or condition caused by gut microbiota malfunction or disturbance wherein the tocopherol contains one, two or more kinds selected from the group consisting of β-tocopherol, γ-tocopherol and δ-tocopherol.

### Item 6:

The use of any one of items 1, 2, or 4 or the method of item 3 or 4, or the composition for use of item 5, wherein the subject is an infant or young child.

### Item 7:

The use of any one of items 1, 2, 4, or 6, or the method of any one of items 3, 4 and 6, or the composition for use of item 5 or 6, or the composition for use of item 5 or 6, wherein the composition is for promoting the assimilation of an oligosaccharide.

### Item 8:

The use of any one of items 1, 2, 4, 6 and 7, or the method of any one of items 3, 4, 6 and 7, or the composition for use of any one of items 5 to 7, wherein the tocopherol comprises (all of) β-tocopherol, γ-tocopherol and δ-tocopherol.

### Item 9:

The use of any one of items 1, 2, 4, and 6 to 8, or the method of any one of items 3, 4, 6 to 8, or the composition for use of any one of items 5 to 8, wherein the mass ratio of the δ-tocopherol content to the β-tocopherol content (δ/β) is 5 or more and/or 200 or less.

### Item 10:

The use according to item 9, or the method of item 9, or the composition for use of item 9, wherein the mass ratio of the δ-tocopherol content to the β-tocopherol content (δ/β) is 10 or more and/or 200 or less.

### Item 11:

The use of any one of items 1, 2, 4, and 6 to 10, or the method of any one of items 3, 4, 6 to 10, or the composition for use of any one of items 5 to 10, wherein the mass ratio of the γ-tocopherol content to the δ-tocopherol content (γ/δ) is 8.8 or less and/or 0.1 or more.

### Item 12:

The use according to item 11, or the method of item 11, or the composition for use of item 11, wherein the mass ratio of the γ-tocopherol content to the δ-tocopherol content (γ/δ) is 3.9 or less and/or 0.1 or more.

### Item 13:

The use of any one of items 1, 2, 4, and 6 to 12, or the method of any one of items 3, 4, 6 to 12, or the composition for use of any one of items 5 to 12, wherein the mass ratio of the total amount of β-tocopherol, γ-tocopherol and δ-tocopherol to the oligosaccharide content is 0.001 to 0.2.

### Item 14:

The use of any one of items 1, 2, 4, and 6 to 13, or the method of any one of items 3, 4, 6 to 13, or the composition for use of any one of items 5 to 13, wherein the total amount of β-tocopherol, γ-tocopherol and δ-tocopherol based on the entire composition is 0.0002 to 0.1 mass %.

### Item 15:

The use of any one of items 1, 2, 4, and 6 to 14, or the method of any one of items 3, 4, 6 to 14, or the composition for use of any one of items 5 to 14, wherein the oligosaccharide content of the entire composition is 0.05 to 10 mass %.

### Item 16:

The use of any one of items 1, 2, 4, and 6 to 15, or the method of any one of items 3, 4, 6 to 15, or the composition for use of any one of items 5 to 15, wherein the composition is for promoting the assimilation of an oligosaccharide having three or more and five or less sugar residues.

### Item 17:

The use of any one of items 1, 2, 4, and 6 to 16, or the method of any one of items 3, 4, 6 to 16, or the composition for use of any one of items 5 to 16, wherein the composition is for promoting the assimilation of a human milk oligosaccharides.

### Item 18:

The use according to item 17, or the method of item 17, or the composition for use of item 17, wherein the human milk oligosaccharides contains one, two or more kinds selected from the group consisting of lacto-N-neotetraose, 2′-fucosyllactose, 3′-sialyllactose, 6′-sialyllactose, difucosyllactose and 3-fucosyllactose, preferably wherein the human milk oligosaccharide is 2′-fucosyllactose or 6′-sialyllactose.

### Item 19:

The use of any one of items 1, 2, 4, and 6 to 18, or the method of any one of items 3, 4, 6 to 18, or the composition for use of any one of items 5 to 18, wherein the composition further contains a human milk oligosaccharides, preferably wherein the human milk oligosaccharides contains one, two or more kinds selected from the group consisting of lacto-N-neotetraose, 2′-fucosyllactose, 3′-sialyllactose, 6′-sialyllactose, difucosyllactose and 3-fucosyllactose, more preferably wherein the human milk oligosaccharide is 2′-fucosyllactose or 6′-sialyllactose.

### Item 20:

The use according to item 19, or the method of item 19, or the composition for use of item 19, wherein the composition contains lacto-N-tetraose, optionally wherein said composition is for promoting the assimilation of a human milk oligosaccharides containing 2′-fucosyllactose.

### Item 21:

The use according to item 19, or the method of item 19, or the composition for use of item 19, wherein the composition contains lacto-N-tetraose and 2′-fucosyllactose.

### Item 22:

The use according to item 19, or the method of item 19, or the composition for use of item 19, wherein the composition contains fructooligosaccharide and 6′-sialyllactose.

### Item 23:

The use of any one of items 1, 2, 4, and 6 to 22, or the method of any one of items 3, 4, 6 to 22, or the composition for use of any one of items 5 to 22, wherein the composition is a nutritional composition.

### Item 24:

The use according to item 23, or the method of item 23, or the composition for use of item 23, wherein the composition is a milk formula, preferably an infant milk formula.

### Item 25:

The use of any one of items 1, 2, 4, and 6 to 24, or the method of any one of items 3, 4, 6 to 24, or the composition for use of any one of items 5 to 24, wherein the composition is added to a composition containing a human milk oligosaccharides before use.

### Item 26:

The use according to item 25, or the method of item 25, or the composition for use of item 25, wherein the composition containing the human milk oligosaccharides is breast milk.

### Item 27:

The use of any one of items 1, 2, 4, and 6 to 26, or the method of any one of items 3, 4, 6 to 26, or the composition for use of any one of items 5 to 26, wherein the composition is for promoting the assimilation of an oligosaccharide by a bifidobacterial strain, preferably wherein the bifidobacterial strain is one, two or more strains selected from the group consisting of Bifidobacterium breve, Bifidobacterium bifidum, Bifidobacterium infantis and Bifidobacterium longum, more preferably wherein the bifidobacterial strain is Bifidobacterium longum subsp. infantis MCC2042, Bifidobacterium infantis M-63 or Bifidobacterium bifidum MCC2030.

### Item 28:

The use of any one of items 1, 2, 4, and 6 to 27, or the method of any one of items 3, 4, 6 to 27, or the composition for use of any one of items 5 to 27, wherein the composition further comprises a bifidobacterial strain, preferably wherein the bifidobacterial strain is one, two or more strains selected from the group consisting of Bifidobacterium breve, Bifidobacterium bifidum, Bifidobacterium infantis and Bifidobacterium longum, more preferably wherein the bifidobacterial strain is Bifidobacterium longum subsp. infantis MCC2042, Bifidobacterium infantis M-63 or Bifidobacterium bifidum MCC2030.

### Item 29:

A nutritional composition comprising an oligosaccharide and a tocopherol,
wherein the tocopherol contains one, two or more kinds selected from the group consisting of β-tocopherol, γ-tocopherol and δ-tocopherol (with the proviso that breast milk is excluded).

### Item 30:

The nutritional composition according to item 29, wherein the mass ratio of the δ-tocopherol content to the β-tocopherol content (δ/β) is 5 or more and/or 200 or less.

### Item 31:

The nutritional composition according to item 30, wherein the mass ratio of the δ-tocopherol content to the β-tocopherol content (δ/β) is 10 or more and/or 200 or less.

### Item 32:

The nutritional composition according to any one of items 29 to 30, wherein the mass ratio of the γ-tocopherol content to the δ-tocopherol content (γ/δ) is 8.8 or less and/or 0.1 or more.

### Item 33:

The nutritional composition according to item 32, wherein the mass ratio of the γ-tocopherol content to the δ-tocopherol content (γ/δ) is 3.9 or less and/or 0.1 or more.

### Item 34:

The nutritional composition according to any one of items 29 to 32, wherein the mass ratio of the total amount of β-tocopherol, γ-tocopherol and δ-tocopherol to the oligosaccharide content is 0.001 to 0.2.

### Item 35:

The nutritional composition according to any one of items 29 to 34, wherein the total amount of β-tocopherol, γ-tocopherol and δ-tocopherol based on the entire composition is 0.0002 to 0.1 mass %.

### Item 36:

The nutritional composition according to any one of items 29 to 35, wherein the oligosaccharide content of the entire composition is 0.05 to 10 mass %.

### Item 37:

The nutritional composition according to any one of items 29 to 36, wherein the oligosaccharide has three or more and five or less sugar residues.

### Item 38:

The nutritional composition according to any one of items 29 to 37, wherein the oligosaccharide contains a human milk oligosaccharides.

### Item 39:

The nutritional composition according to item 38, wherein the human milk oligosaccharides contains one, two or more kinds selected from the group consisting of lacto-N-neotetraose, lacto-N-tetraose, 2′-fucosyllactose, 3′-sialyllactose, 6′-sialyllactose, difucosyllactose and 3-fucosyllactose, more preferably wherein the human milk oligosaccharide is 2′-fucosyllactose or 6′-sialyllactose.

### Item 40:

The nutritional composition according to item 37, wherein the oligosaccharide contains lacto-N-tetraose and 2′-fucosyllactose.

### Item 41:

The nutritional composition according to item 37, wherein the oligosaccharide contains fructooligosaccharide and 6′-sialyllactose.

### Item 42:

The nutritional composition according to any one of items 29 to 41 which is for promoting the growth of a bifidobacterial strain.

### Item 43:

The nutritional composition according to item 42, wherein the bifidobacterial strain is one, two or more strains selected from the group consisting of Bifidobacterium breve, Bifidobacterium bifidum, Bifidobacterium infantis and Bifidobacterium longum, more preferably wherein the bifidobacterial strain is Bifidobacterium longum subsp. infantis MCC2042, Bifidobacterium infantis M-63 or Bifidobacterium bifidum MCC2030.

### Item 44:

The nutritional composition according to any one of items 29 to 43 which further contains a bifidobacterial strain, preferably wherein the bifidobacterial strain is one, two or more strains selected from the group consisting of Bifidobacterium breve, Bifidobacterium bifidum, Bifidobacterium infantis and Bifidobacterium longum, more preferably wherein the bifidobacterial strain is Bifidobacterium longum subsp. infantis MCC2042, Bifidobacterium infantis M-63 or Bifidobacterium bifidum MCC2030.

### Item 45:

The nutritional composition according to any one of items 29 to 44 which is a formula, preferably an infant formula.

### <First Aspect: Composition for Promoting Assimilation of Oligosaccharide>

The composition of the aspect contains a tocopherol as an active ingredient.

The tocopherol contains one, two or more kinds selected from the group consisting of β-tocopherol, γ-tocopherol and δ-tocopherol. Preferably, all the three kinds of β-tocopherol, γ-tocopherol and δ-tocopherol are contained. Moreover, the composition of the aspect may further contain α-tocopherol.

In the composition of the aspect, the mass ratio of the δ-tocopherol content to the β-tocopherol content (δ/β) is preferably 5 or more, more preferably 10 or more. The upper limit of δ/β is not particularly limited but is generally 200 or less, such as 150 or less, 100 or less, 90, 80, 70, 60, 50, 40, 30, 25 or 20 or less. The mass ratio of the δ-tocopherol content to the β-tocopherol content (δ/β) is preferably between 5 to 25, more preferably between 10 to 25, and even more preferably between 10 to 16.

When δ/β is in the range, the effect of promoting the assimilation of an oligosaccharide by an intestinal bacterium is obtained more easily.

In the composition of the aspect, the mass ratio of the γ-tocopherol content to the δ-tocopherol content (γ/δ) is preferably 9 or less, 8.9 or less or 8.8 or less, more preferably 4 (e.g. 4.0) or less, or 3.9 or less. The lower limit of γ/δ is not particularly limited but is generally 0.1 or more, such as 0.5 or more, 1 or more, 1.5, 1.6, 1.7, 1.8, 1.9, 2 or 2.0 or more. The mass ratio of the γ-tocopherol content to the δ-tocopherol content (γ/δ) is preferably between 1 to 9 (or 8.9 or 8.7), more preferably between 1 to 4 (4.0 or 3.9), and even more preferably between 2 (2.1) to 4 (3.9).

When γ/δ is in the range, the effect of promoting the assimilation of an oligosaccharide by an intestinal bacterium is obtained more easily.

The Table 1 below shows the tocopherol patterns used herein, based on which ratios were determined for use in the presentation invention:

TABLE 1

| | Pattern 1 | Pattern 2 | Pattern 3 | Pattern 4 |
|---|---|---|---|---|
| β | 1 | 1 | 1 | 1 |
| γ | 24.5 | 33 | 39 | 44 |
| δ | 24.5 | 16 | 10 | 5 |
| γ/δ | 1 | 2.1 | 3.9 | 8.8 |
| δ/β | 24.5 | 16 | 10 | 5 |
| γ/β | 24.5 | 33 | 39 | 44 |

The use of patterns 1 to 4 is envisaged, as is the use of any range between patterns 1 to 4, preferably between patterns 1 to 3, and more preferably between patterns 2 to 3.

In accordance with the above, a (mass) ratio of β:γ:δ in the composition preferably is β:γ:δ=1:24.5-44:5-24.5, more preferably β:γ:δ=1:25-39:10-24.5, most preferably β:γ:δ=1:33-39:10-16.

The total amount of β-tocopherol, γ-tocopherol and δ-tocopherol based on the entire composition (per solid content) in the composition of the aspect is not particularly limited.

The total amount in the composition of the aspect is adjusted or applied in such a manner that the mass ratio of the total amount of β-tocopherol, γ-tocopherol and δ-tocopherol to the amount of the oligosaccharide as the subject of promotion of assimilation becomes preferably 0.001 to 0.2, more preferably 0.004 to 0.2, further preferably 0.01 to 0.2.

The composition of the aspect can promote direct or indirect assimilation of an oligosaccharide by an intestinal bacterium because β-tocopherol, γ-tocopherol and/or δ-tocopherol functions as an active ingredient.

In general, an intestinal bacterium which has assimilated an oligosaccharide produces various beneficial substances, and the composition of the aspect promotes the production thereof and thus can contribute to enhancement of the health of the host of the intestinal bacterium.

Moreover, in general, an intestinal bacterium assimilates an oligosaccharide for the growth, and when the assimilation of an oligosaccharide is promoted, the intestinal bacterium whose growth is promoted with the oligosaccharide becomes dominant in the intestines as a result. Accordingly, the composition of the aspect can also support and improve the effect of the oligosaccharide as a prebiotic material.

That an oligosaccharide is assimilated can be confirmed, for example, when the oligosaccharide decreases with time after culturing an intestinal bacterium in the presence of the oligosaccharide. More specifically, it can be confirmed when the residual rate of the amount of the oligosaccharide in the culture system after a certain period of time is smaller than the amount of the oligosaccharide in the culture system at a certain point of time (for example, at the beginning of the culture or the like).

The “promotion of the assimilation” in the present specification means that the degree of the assimilation of the oligosaccharide with the application of the composition of the aspect is larger than that without the application, in other words, the residual rate of the oligosaccharide after a certain period of time is smaller. The degree of the decrease in the residual rate is not particularly limited but is a residual rate that is preferably 0.9 times or less, more preferably 0.8 times or less, further preferably 0.7 times or less the residual rate without the application of the composition of the aspect. Alternatively, the residual rate after seven hours, 10 hours or 24 hours of culture based on the value at the beginning of culture may be preferably 70 mass % or less, more preferably 50 mass % or less, further preferably 20 mass % or less.

Moreover, the “promotion of the assimilation” may mean that the speed of decrease in the oligosaccharide through assimilation increases when the composition of the aspect is applied compared to the case without the application. The degree of the increase in the speed is not particularly limited but is preferably 10% or more, more preferably 20% or more, further preferably 50% or more higher than the speed of decrease without the application of the composition of the aspect. The increase in the decrease speed can be confirmed, for example, when the time required until the residual rate based on the value at the beginning of culture reaches a certain value becomes shorter when the composition of the aspect is applied compared to the time without the application. Alternatively, it can be determined that assimilation is promoted when the culture period required until the residual rate based on the value at the beginning of culture becomes 20 mass % or less is preferably 24 hours or shorter, more preferably 18 hours or shorter, preferably 12 hours or shorter.

That the oligosaccharide is assimilated can also be confirmed when the growth of an intestinal bacterium is promoted after culturing the intestinal bacterium in the presence of the oligosaccharide. For the promotion of the growth of the intestinal bacterium, please see the explanation in the second aspect described below.

The oligosaccharide whose assimilation is promoted in the aspect is assimilated by an intestinal bacterium. The intestinal bacterium may be a strain of Bifidobacterium, a strain of Enterococcus, a strain of Streptococcus, a strain of Escherichia, a strain of Staphylococcus, a strain of Turicibacter, a strain of Clostridium, a strain of Ruminococcus, a strain of Veillonella, a strain of Bacteroides, a strain of Parabacteroides, a strain of Lacticaseibacillus, a strain of Lactiplantibacillus, a strain of Limosilactobacillus, a strain of Levilactobacillus, a strain of Ligilactobacillus, a strain of Latilactobacillus, a strain of Blautia or the like.

The oligosaccharide whose assimilation is promoted in the aspect is not particularly limited. Here, the oligosaccharide refers to an oligomer of saccharides in which di- or higher saccharides are bonded through a glycoside bond. The oligosaccharide in the aspect has two or more, preferably two or more and 10 or less, especially preferably three or more and five or less sugar residues which are bonded to each other through a glycoside bond.

The oligosaccharide may be specifically raffinose, lactulose, maltotriose, stachyose, galactooligosaccharide, fructooligosaccharide, kestose, nistose, a soybean oligosaccharide, lactosucrose, xylooligosaccharide, isomaltooligosaccharide, coffee bean mannooligosaccharide, gluconic acid, polydextrose, inulin or the like.

Of these, preferable oligosaccharides having three or more and five or less sugar groups are maltotriose, stachyose, galactooligosaccharide, fructooligosaccharide, kestose, nistose, a human milk oligosaccharides and the like.

As the oligosaccharide whose assimilation is promoted in the aspect, a human milk oligosaccharides is also especially preferable.

The human milk oligosaccharides is not particularly limited as long as the human milk oligosaccharides is an oligosaccharide which is generally contained in human milk and may be a fucosylated oligosaccharide, a sialylated oligosaccharide, an acetylated oligosaccharide, a galactosyllactose or the like.

The fucosylated oligosaccharide may be 2′-fucosyllactose, lacto-N-fucopentaose-I, lacto-N-fucopentaose-II, lacto-N-fucopentaose-III or the like.

The sialylated oligosaccharide may be 3′-sialyllactose, 6′-sialyllactose, difucosyllactose, 3-fucosyllactose, lactodifucotetraose, difucosyllacto-N-tetraose, sialyllacto-N-tetraose a, sialyllacto-N-tetraose b, sialyllacto-N-tetraose c, disialyllacto-N-tetraose or the like.

The acetylated oligosaccharide may be lacto-N-neotetraose, lacto-N-hexaose or the like.

The galactosyllactose may be 3′-galactosyllactose, 4′-galactosyllactose, 6′-galactosyllactose or the like.

Of these, in view of the effect of promoting assimilation, one, two or more kinds selected from lacto-N-neotetraose, 2′-fucosyllactose, 3′-sialyllactose, 6′-sialyllactose, difucosyllactose and 3-fucosyllactose are especially preferable.

The composition of the aspect may further contain a human milk oligosaccharides.

The human milk oligosaccharides may be the fucosylated oligosaccharide, the sialylated oligosaccharide or the acetylated oligosaccharide described above or the like and is more preferably one, two or more kinds selected from lacto-N-neotetraose, lacto-N-tetraose, 2′-fucosyllactose, 3′-sialyllactose, 6′-sialyllactose, difucosyllactose and 3-fucosyllactose in view of the effect of promoting assimilation. More preferably the human milk oligosaccharide is 2′-fucosyllactose or 6′-sialyllactose (as sole HMOs or sole oligosaccharides).

In particular, the composition of the aspect preferably contains lacto-N-tetraose together with the certain tocopherol and can be especially preferably applied to use for promoting the assimilation of a human milk oligosaccharides containing 2′-fucosyllactose in this case because the assimilation of 2′-fucosyllactose can be further promoted. That is, as an especially preferable aspect of the aspect, a composition for promoting the assimilation of a human milk oligosaccharides containing a tocopherol and lacto-N-tetraose in which the tocopherol contains one, two or more kinds selected from the group consisting of β-tocopherol, γ-tocopherol and δ-tocopherol and in which the human milk oligosaccharides contains 2′-fucosyllactose is disclosed. In a preferred aspect, the composition comprises a human milk oligosaccharide as defined herein, preferably 2′-fucosyllactose or 6′-sialyllactose, and lacto-N-tetraose (as sole oligosaccharides). In a particularly preferred aspect, the composition comprises 2′-fucosyllactose and lacto-N-tetraose (as sole oligosaccharides).

The human milk oligosaccharides contained in the composition of the aspect may be a purified one or a mixture as long as the effects of the aspect are not impaired.

The composition of the aspect may further contain an oligosaccharide other than the human milk oligosaccharides. The oligosaccharide may be any of those described above, and in particular, fructooligosaccharide is also preferably contained together with the certain tocopherol. In this case, because the assimilation of 6′-sialyllactose can be further promoted, the composition can be especially preferably applied to use for promoting the assimilation of a human milk oligosaccharides containing 6′-sialyllactose. That is, as an especially preferable aspect of the aspect, a composition for promoting the assimilation of a human milk oligosaccharides containing a tocopherol and fructooligosaccharide in which the tocopherol contains one, two or more kinds selected from the group consisting of β-tocopherol, γ-tocopherol and δ-tocopherol and in which the human milk oligosaccharides contains 6′-sialyllactose is disclosed.

When the oligosaccharide (including the human milk oligosaccharides) is contained in the composition of the aspect, the total amount thereof based on the entire composition (per solid content) is preferably 0.05 to 10 mass %, more preferably 0.1 to 10 mass %, further preferably 0.2 to 10 mass %.

Moreover, when the oligosaccharide (including the human milk oligosaccharides) is contained in the composition of the aspect, the mass ratio of the total amount of β-tocopherol, γ-tocopherol and δ-tocopherol to the oligosaccharide content is preferably 0.001 to 0.2, more preferably 0.004 to 0.2, further preferably 0.01 to 0.2.

The aspect can be rephrased as use of one, two or more tocopherols selected from the group consisting of β-tocopherol, γ-tocopherol and δ-tocopherol in the manufacture of a composition for promoting the assimilation of an oligosaccharide.

The aspect can also be rephrased as use of one, two or more tocopherols selected from the group consisting of β-tocopherol, γ-tocopherol and δ-tocopherol in the promotion of the assimilation of an oligosaccharide.

The aspect can also be rephrased as one, two or more tocopherols selected from the group consisting of β-tocopherol, γ-tocopherol and δ-tocopherol which is used for promoting the assimilation of an oligosaccharide.

The aspect can also be rephrased as a method for promoting the assimilation of an oligosaccharide including administering one, two or more tocopherols selected from the group consisting of β-tocopherol, γ-tocopherol and δ-tocopherol to a subject. In the method, an oligosaccharide is preferably administered to the subject at the same time with or before or after the administration of the tocopherol.

Here, the subject is not particularly limited as long as the subject is an animal, but the subject is generally a human. In this regard, “to administer to a subject” may be synonymous with “to cause a subject to take”. The intake may be voluntary (free intake) or forced (forced intake). That is, the administration step may be specifically, for example, a step of blending one, two or more tocopherols selected from the group consisting of β-tocopherol, γ-tocopherol and δ-tocopherol in a food, a drink or feed for supplying to a subject and thus causing the subject to freely take the food, the drink or the feed.

### <Second Aspect: Nutritional Composition>

As described above, in the composition of the first aspect, β-tocopherol, γ-tocopherol and/or δ-tocopherol functions as an active ingredient, and the composition promotes the assimilation of an oligosaccharide by an intestinal bacterium and promotes the growth of the intestinal bacterium. Accordingly, the composition of the first aspect is suitable as a nutritional composition when an oligosaccharide is contained together.

That is, the present specification provides a nutritional composition containing an oligosaccharide and a tocopherol as a second aspect. In this regard, however, breast milk itself is not included in the nutritional composition of the aspect.

The nutritional composition of the aspect contains an oligosaccharide and a tocopherol.

The explanation for the kinds of the tocopherol in the aspect is in accordance with the explanation given in the first aspect.

Preferable ranges of the mass ratio of the δ-tocopherol content to the β-tocopherol content (δ/β) and the mass ratio of the γ-tocopherol content to the δ-tocopherol content (γ/δ) in the aspect are in accordance with the respective explanations given in the first aspect.

Within the ranges, the effect of promoting the growth of a bifidobacterial strain is more easily obtained.

In the nutritional composition of the aspect, the total amount of β-tocopherol, γ-tocopherol and δ-tocopherol based on the entire composition is preferably 0.0002 to 0.1 mass %, more preferably 0.001 to 0.04 mass %, further preferably 0.002 to 0.04 mass % more preferably about 0.03 mass %, particularly preferably 0.028 mass %.

When the total amount is in the range, the effect of promoting the growth of a bifidobacterial strain is more easily obtained.

The explanation for the kinds of the oligosaccharide (including the human milk oligosaccharides) in the aspect is in accordance with the explanation for the assimilated oligosaccharide and the oligosaccharide which can be contained in the composition in the first aspect, and any one, two or more kinds thereof can be used. In this regard, however, lacto-N-tetraose is also added to the preferable examples of the acetylated oligosaccharide listed as the human milk oligosaccharides.

As the oligosaccharide that the composition of the aspect contains, a combination of lacto-N-tetraose and 2′-fucosyllactose is especially preferably contained. In this case, because lacto-N-tetraose together with the tocopherol promotes the assimilation of 2′-fucosyllactose by a bifidobacterial strain, the effect of promoting the growth of the strain is more easily obtained.

Moreover, as the oligosaccharide that the composition of the aspect contains, a combination of fructooligosaccharide and 6′-sialyllactose is also especially preferably contained. In this case, because fructooligosaccharide together with the tocopherol promotes the assimilation of 6′-sialyllactose by a bifidobacterial strain, the effect of promoting the growth of the strain is more easily obtained.

A preferable range of the mass ratio of the total amount of β-tocopherol, γ-tocopherol and δ-tocopherol to the oligosaccharide content in the aspect is in accordance with the explanation for the case in which the oligosaccharide is contained in the first aspect.

Within the range, the effect of promoting the growth of a bifidobacterial strain is more easily obtained.

In the nutritional composition of the aspect, a preferable range of the total oligosaccharide content is in accordance with the explanation for the case in which the oligosaccharide is contained in the first aspect.

Within the range, the effect of promoting the growth of a bifidobacterial strain is more easily obtained.

The nutritional composition of the aspect can promote the growth of an intestinal bacterium, especially a bifidobacterial strain (Bifidobacterium). More specifically, in general, the oligosaccharide is assimilated by a bifidobacterial strain and promotes the growth thereof, and because β-tocopherol, γ-tocopherol and/or δ-tocopherol further improves the growth promotion effect, the nutritional composition can be an excellent prebiotic material.

The bifidobacterial strain whose growth is promoted by the nutritional composition of the aspect is not particularly limited, but examples thereof include Bifidobacterium longum (reclassified as Bifidobacterium longum subsp. longum), Bifidobacterium breve, Bifidobacterium infantis (reclassified as Bifidobacterium longum subsp. infantis), Bifidobacterium bifidum, Bifidobacterium adolescentis, Bifidobacterium animalis, Bifidobacterium pseudolongum, Bifidobacterium reuteri, Bifidobacterium catenulatum, Bifidobacterium pseudocatenulatum, Bifidobacterium lactis, Bifidobacterium longum subsp. suis and the like. Of these, Bifidobacterium longum, Bifidobacterium breve, Bifidobacterium infantis and Bifidobacterium bifidum are four species which are especially important for the health of the enteric microbiota in the infancy and the early childhood (so-called “infant-type bifidobacteria”), and the growth thereof is preferably promoted.

The “growth” of a strain in the present specification includes an increase in the count of the strain, and the “promotion of the growth” means that the degree of the increase is larger when the nutritional composition of the aspect is applied compared to the degree without the application. That is, an effect of increasing the bacterial count of the intestinal bacterium such as the bifidobacterial strain with the in vivo or in vitro application of the nutritional composition of the aspect, as compared to the bacterial count without the application, is obtained. The degree of the increase in the bacterial count is not particularly limited but means that the bacterial count becomes preferably 1.1 times or more, more preferably 1.5 times or more, further preferably three times or more the bacterial count of the strain without the application of the nutritional composition of the aspect.

The increase in the bacterial count can be confirmed by directly measuring the bacterial count or with, for example, an increase in the value obtained by measuring the turbidity (e.g. absorbance at 600 nm (OD600)) of the culture medium of the strain or by measuring the amount of a short-chain fatty acid such as acetic acid of the medium or the gastrointestinal content of an animal such as a human which has taken a composition of the strain, a decrease in the measured pH value of the medium or the like.

The “growth” also includes an increase in the proportion of the strain in the enteric microbiota, and the “promotion of the growth” means that the degree of the increase is larger when the nutritional composition of the aspect is applied compared to the degree without the application. That is, increasing the proportion of the bifidobacterial strain in the gastrointestinal tract of an animal such as a human which has taken the nutritional composition of the aspect is included. Here, the “proportion” can be rephrased as the “occupying ratio” to the entire bacterial group detected from the enteric microbiota. Regarding the “increase in the proportion”, as long as the proportion of the bifidobacterial strain in the enteric microbiota increases, the proportion of another strain in the enteric microbiota may increase or decrease at the same time.

The degree of the increase in the proportion is not particularly limited but means that the proportion is preferably 2% or more, more preferably 5% or more, further preferably 20% or more higher than the proportion of the strain without the application of the nutritional composition of the aspect.

The “promotion of the growth” may also be an increase in the speed of the increase in the count of the strain or in the speed of the increase in the proportion of the strain in the enteric microbiota. The degree of the increase in such a speed is not particularly limited but means that the speed is preferably 10% or more, more preferably 20% or more, further preferably 50% or more higher than the speed of the strain without the application of the nutritional composition of the aspect.

The increase in the speed can be confirmed, for example, when the time required until the bacterial count or the proportion reaches a certain value is shorter when the nutritional composition of the aspect is applied compared to the time without the application.

As described above, because the nutritional composition of the aspect can promote the growth of a bifidobacterial strain, the composition preferably contains a bifidobacterial strain together with the oligosaccharide and one, two or more tocopherols selected from the group consisting of β-tocopherol, γ-tocopherol and δ-tocopherol. Here, the bifidobacterial strain contained in the nutritional composition may be Bifidobacterium longum (Bifidobacterium longum subsp. longum), Bifidobacterium breve, Bifidobacterium infantis (Bifidobacterium longum subsp. infantis), Bifidobacterium bifidum, Bifidobacterium adolescentis, Bifidobacterium animalis, Bifidobacterium pseudolongum, Bifidobacterium reuteri, Bifidobacterium catenulatum, Bifidobacterium pseudocatenulatum, Bifidobacterium lactis, Bifidobacterium longum subsp. suis or the like and is not particularly limited. Of these, Bifidobacterium longum, Bifidobacterium breve, Bifidobacterium infantis and Bifidobacterium bifidum, which are especially important in infants and young children, are preferably contained.

As Bifidobacterium longum, Bifidobacterium longum NITE BP-02621 (other names: BB536 or Bifidobacterium longum subsp. longum ATCC BAA-999) can be used. Bifidobacterium longum BB536 was deposited for an international deposit under the Budapest Treaty to NPMD on Jan. 26, 2018 with an accession number of NITE BP-02621. The identical strain thereof, Bifidobacterium longum subsp. longum ATCC BAA-999 (number: ATCC BAA-999), can be obtained from American Type Culture Collection (ATCC: 10801 University Boulevard, Manassas, VA 20110, United States of America) as ATCC BAA-999 (for example, see JP-A-2012-223134 and the like).

Moreover, as Bifidobacterium longum, Bifidobacterium longum subsp. longum ATCC 15707, which can be obtained from ATCC, can be used.

Bifidobacterium breve may be Bifidobacterium breve M-16V. Bifidobacterium breve M-16V was deposited for an international deposit under the Budapest Treaty on Jan. 26, 2018 to NITE Patent Microorganisms Depositary, National Institute of Technology and Evaluation (NPMD) (Room 122, 2-5-8 Kazusakamatari, Kisarazu-shi, Chiba 292-0818) with an accession number of NITE BP-02622. For example, “Bifidobacterium breve M-16V” manufactured by Morinaga Milk Industry Co., Ltd., which can be obtained as a commercial product, may also be used.

Bifidobacterium breve may also be Bifidobacterium breve MCC1274. Bifidobacterium breve MCC1274 was deposited for an international deposit under the Budapest Treaty on Aug. 25, 2009 to International Patent Organism Depositary, National Institute of Advanced Industrial Science and Technology (current International Patent Organism Depositary (IPOD), National Institute of Technology and Evaluation (Room 120, 2-5-8 Kazusakamatari, Kisarazu-shi, Chiba 292-0818) with an accession number of FERM BP-11175.

Bifidobacterium infantis may be Bifidobacterium longum subsp. infantis MCC2042. Bifidobacterium longum subsp. infantis MCC2042 was deposited for an international deposit under the Budapest Treaty on Nov. 20, 2019 to NPMD with an accession number of NITE BP-03068. Bifidobacterium infantis may also be Bifidobacterium infantis M-63. Bifidobacterium infantis M-63 was deposited for an international deposit under the Budapest Treaty on Jan. 26, 2018 to NPMD with an accession number of NITE BP-02623.

As Bifidobacterium infantis, Bifidobacterium longum subsp. infantis ATCC 15697, which can be obtained from ATCC, can also be used.

Bifidobacterium bifidum may be Bifidobacterium bifidum MCC2030. Bifidobacterium bifidum MCC2030 was deposited for an international deposit under the Budapest Treaty on Nov. 8, 2019 to NPMD with an accession number of NITE BP-03058.

Bifidobacterium bifidum may be Bifidobacterium bifidum MCC1092. Bifidobacterium bifidum MCC1092 was deposited for an international deposit under the Budapest Treaty on Feb. 21, 2017 to NPMD with an accession number of NITE BP-02429.

Bifidobacterium bifidum may also be Bifidobacterium bifidum MCC1319. Bifidobacterium bifidum MCC1319 was deposited for an international deposit under the Budapest Treaty on Feb. 21, 2017 to NPMD with an accession number of NITE BP-02431.

Bifidobacterium bifidum may also be Bifidobacterium bifidum MCC1868. Bifidobacterium bifidum MCC1868 was deposited for an international deposit under the Budapest Treaty on Feb. 21, 2017 to NPMD with an accession number of NITE BP-02432.

Bifidobacterium bifidum may also be Bifidobacterium bifidum MCC1870. Bifidobacterium bifidum MCC1870 was deposited for an international deposit under the Budapest Treaty on Feb. 21, 2017 to NPMD with an accession number of NITE BP-02433.

Bifidobacterium longum subsp. infantis MCC2042, Bifidobacterium infantis M-63 and Bifidobacterium bifidum MCC2030 are preferred herein.

The strains specified by the bacterial names exemplified above are not limited to the strains deposited or registered at the certain organizations under the bacterial names themselves (also called “deposited strains” below for the convenience of explanation) but include substantially equivalent strains thereof (also called “derived strains” or “induced strains”). That is, the strains are not limited to the strains deposited to the depositaries with the accession numbers themselves but include substantially equivalent strains thereof. A “substantially equivalent strain of a deposited strain” of the strains is a strain belonging to the identical species to that of the deposited strain in which the base sequence of 16SrRNA gene has an identity of preferably 99.86% or more, more preferably 99.93% or more, further preferably 100% to the base sequence of 16SrRNA gene of the deposited strain and which preferably has the identical bacteriological properties to those of the deposited strain. The substantially equivalent strains of a deposited strain of the strains may be, for example, strains derived from the deposited strain as the parent strain. The derived strains include a strain bred from the deposited strain and a strain naturally generated from the deposited strain. Breeding methods include modification by the genetic engineering technique and modification by mutagenesis. The mutagenesis includes X-ray irradiation, ultraviolet irradiation and treatment with a mutagen such as N-methyl-N′-nitro-N-nitrosoguanidine, ethyl methanesulfonate and methyl methanesulfonate. The strain naturally generated from the deposited strain is a strain which is naturally generated from the deposited strain during use. Such a strain is a mutant naturally generated during culture of the deposited strain (for example, passaging culture). The derived strains may be constructed with a kind of modification or constructed with two or more kinds of modification.

As bacterial cells of the bifidobacterial strain contained in the nutritional composition of the aspect, a commercial product may be used, and one appropriately produced and obtained may be used.

Moreover, bacterial cells of the bifidobacterial strain contained in the nutritional composition of the aspect can be easily obtained by culturing any of the bifidobacterial strains described above. The culture method is not particularly restricted as long as the bifidobacterial strain can grow. As the culture method, for example, a method which is generally used for culturing the bifidobacterial strain can be used directly or with appropriate modification. The culture temperature may be, for example, 25 to 50° C. and is preferably 35 to 42° C. The culture can be conducted preferably under anaerobic conditions and can be conducted, for example, while flowing an anaerobic gas such as carbon dioxide gas. The culture can also be conducted under microaerophilic conditions such as static liquid culture. The culture can be conducted, for example, until the bifidobacterial strain grows to a desired degree or for 5 to 24 hours.

The medium used for the culture is not particularly restricted as long as the bifidobacterial strain can grow. As the medium, for example, a medium which is generally used for culturing the bifidobacterial strain can be used directly or with appropriate modification. That is, as a carbon source, for example, saccharides such as galactose, glucose, fructose, mannose, cellobiose, maltose, lactose, sucrose, trehalose, starch, starch hydrolysate and molasses can be used depending on the assimilation properties. As a nitrogen source, for example, ammonia and ammonium salts and nitrates such as ammonium sulfate, ammonium chloride and ammonium nitrate can be used. Moreover, as an inorganic salt, for example, sodium chloride, potassium chloride, potassium phosphate, magnesium sulfate, calcium chloride, calcium nitrate, manganese chloride, ferrous sulfate and the like can be used. Furthermore, organic components such as peptone, soybean powder, a defatted soybean cake, meat extract and yeast extract and antioxidants such as ascorbic acid, its derivatives, salts thereof, α-lipoic acid, its derivatives and salts thereof may also be used. Specific examples of the medium which is generally used for culturing the bifidobacterial strain include reinforced clostridial medium, MRS medium (de Man, Rogosa, and Sharpe medium), mMRS medium (modified MRS medium), TOSP medium (TOS propionate medium) and TOSP Mup medium (TOS propionate mupirocin medium).

As the bifidobacterial strain which can be contained in the nutritional composition of the aspect, bacterial cells thereof or a fraction containing the same can be used without particular restriction. That is, as the bifidobacterial strain, for example, a culture obtained through culture may be used directly. Alternatively, a diluted or concentrated culture may be used, or bacterial cells collected from the culture may also be used. Various additional operations such as heating and lyophilization can be conducted after the culture as long as the enteric microbiota improvement effect is not impaired. The additional operation is preferably an operation in which the viability of the bacterial cells is high. That is, specifically, the bifidobacterial strain which can be contained in the nutritional composition of the aspect may be a culture of the bifidobacterial strain, bacterial cells collected from the culture or a treated product thereof, and the treated product may be a diluted product, a concentrated product, a dried product or the like. The bacterial cells are preferably used generally in the form containing living bacterial cells. For example, the bacterial cells may be composed of living bacterial cells or may be a mixture of living bacterial cells and dead bacterial cells.

In the nutritional composition of the aspect, the bifidobacterial strain contained in the composition contains at least the living strain, and the living bifidobacterial strain is contained in the composition in the total amount of preferably 1×10⁴ to 1×10¹³ cfu/g or 1×10⁴ to 1×10¹³ cfu/mL, more preferably 1×10⁵ to 1×10¹² cfu/g or 1×10⁵ to 1×10¹² cfu/mL, further preferably 1×10⁶ to 1×10¹¹ cfu/g or 1×10⁶ to 1×10¹¹ cfu/mL.

Moreover, in the nutritional composition of the aspect, the total living bacterial count of the bifidobacterial strain contained in the composition, per 1 g of the oligosaccharide contained in the composition, is preferably 1×10⁸ to 1×10¹¹ cfu, more preferably 1×10⁸ to 1×10¹⁰ cfu, further preferably 1×10⁸ to 3×10⁹ cfu.

As long as the living strain is contained, the dead strain may also be contained.

Here, cfu refers to the colony forming unit. In the present specification, for example, a value obtained by culturing the strain on a solid medium containing 10 mass % reduced skim milk powder at 38° C. can be used.

The ranges may be generally ranges for the amount for distribution as a nutritional composition or the amount for oral intake.

As described above, the nutritional composition of the aspect can promote the growth of a bifidobacterial strain such as infant-type bifidobacteria and thus can be preferably applied for use for promoting the growth of a bifidobacterial strain.

Here, the bifidobacterial strain as the target of growth is not limited to the bifidobacterial strain contained in the nutritional composition of the aspect, and bifidobacterial strains in the gastrointestinal tract of an animal such as a human which has orally taken the nutritional composition are also included.

Accordingly, the nutritional composition of the aspect can be used for improving the enteric microbiota. Here, the “improvement of the microbiota” includes increasing the bacterial count of the bifidobacterial strain, especially infant-type bifidobacteria, in the enteric microbiota or the proportion thereof. Moreover, the “improvement of the microbiota” may include increasing the proportion of another beneficial bacterium in the enteric microbiota and decreasing the proportion of a harmful bacterium in the enteric microbiota as long as the proportion of the bifidobacterial strain in the enteric microbiota increases. The other beneficial bacterium may be a lactic acid bacterium or the like. The harmful bacterium may be Clostridium perfringens, a Salmonella strain, Staphylococcus aureus, pathogenic Escherichia coli or the like.

The “proportion” can be rephrased as the “occupying ratio” to the entire bacterial group detected from the enteric microbiota.

The nutritional composition of the aspect can be useful for a subject whose gut flora is to be improved. The terms “gut flora”, “gut microbiota” and “gut microbiome” are used interchangeably herein. The “gut flora” refers to the microorganisms, including bacteria, archaea, fungi, and viruses that live in the digestive tracts of animals, specifically humans. The “gut flora” includes enteric bacteria, specifically beneficial (non-pathogenic) enteric bacteria. The subject in this context preferably is a healthy subject, such as, but not limited, to an infant, young child, child, youth or adult.

In accordance with the above, the invention relates in one aspect to the use of a composition for gut flora improvement in a subject.

In one aspect, the invention relates to a method for gut flora improvement, wherein a composition comprising a tocopherol is administered to a subject, wherein the tocopherol contains one, two or more kinds selected from the group consisting of β-tocopherol, γ-tocopherol and δ-tocopherol. It is envisaged herein that the tocopherol is the active agent. In other aspects, the tocopherol and the oligosaccharides (specifically HMOs) are the active agent. In further aspect, the tocopherol, the oligosaccharides (specifically HMOs) and the bifidobacterial are the active agent (s).

The term “gut flora improvement” can refer to maintaining or improving a healthy intestinal tract or maintaining or improving gut health or intestinal health. The term “gut flora improvement” can accordingly include or refer to one or more of the following: “for efficient use of prebiotics”, “for helping the functions of oligosaccharides which help the health of babies”, “for health of the gut condition of infants and young children”, “those who wish to increase bifidobacteria”, “for increasing bifidobacteria which help the health of babies”, “improving the enteric microbiota”, “improving the intestinal microbiota”, for “promoting the assimilation of an oligosaccharide”, for “promoting the growth of a bifidobacterial strain, such as infant-type bifidobacterial” and the like.

The nutritional composition of the aspect can be useful for a subject with a disease or a pathological condition which can be prevented or improved through improvement of the gut microbiota or for a subject with a disease or a pathological condition which is caused by deterioration of the gut microbiota. “improvement” can mean the therapeutic treatment of a disease or condition, like ameliorating symptoms of the disease.

In accordance with the above, the invention relates in one aspect to a composition comprising a tocopherol as disclosed herein for use in preventing or treating a disease or condition caused by gut microbiota malfunction or disturbance,
wherein the tocopherol contains one, two or more kinds selected from the group consisting of β-tocopherol, γ-tocopherol and δ-tocopherol. The invention relates in one aspect to a composition comprising a tocopherol as disclosed herein for use in preventing or treating gut microbiota malfunction or disturbance,
wherein the tocopherol contains one, two or more kinds selected from the group consisting of β-tocopherol, γ-tocopherol and δ-tocopherol.

Gut microbiota malfunction or disturbance can relate to a malfunction or disturbance of gut health or a malfunction of the intestinal flora. This in turn can lead to/result in diseases or conditions which are disclosed further below. In this sense, the composition can be used for intestinal regulation.

For example, the nutritional composition can be for improving/promoting immune system especially in a subject with suppressed/decreased immune response/immune system (such as a subject suffering or having suffered from an infection (e.g. a cold, an influenza, a viral and/or a bacterial infection. The nutritional composition can also be for decreasing hyperimmunity (e.g. for use in treating or preventing allergy). in this sense, the composition can be used for immunomodulation.

For example, the nutritional composition can be for intestinal regulation, immunomodulation, antiallergic use, protection against bacterial or viral infection, reduction of oxidative stress, prevention or improvement of diarrhea, prevention or improvement of constipation, inflammatory bowel disease, prevention of colon cancer or the like. Protection as used herein primarily means prevention, but can also include therapeutic treatment, e.g. amelioration of symptoms.

The aspect can be rephrased as use of an oligosaccharide and one, two or more tocopherols selected from the group consisting of β-tocopherol, γ-tocopherol and δ-tocopherol in the manufacture of a composition for promoting the growth of a bifidobacterial strain.

The aspect can also be rephrased as use of an oligosaccharide and one, two or more tocopherols selected from the group consisting of β-tocopherol, γ-tocopherol and δ-tocopherol in the promotion of the growth of a bifidobacterial strain.

The aspect can also be rephrased as an oligosaccharide and one, two or more tocopherols selected from the group consisting of β-tocopherol, γ-tocopherol and δ-tocopherol which are used for promoting the growth of a bifidobacterial strain.

The aspect can also be rephrased as a method for promoting the growth of a bifidobacterial strain including administering an oligosaccharide and one, two or more tocopherols selected from the group consisting of β-tocopherol, γ-tocopherol and δ-tocopherol to a subject.

Here, the subject is not particularly limited as long as the subject is an animal, but the subject is generally a human. In this regard, “to administer to a subject” may be synonymous with “to cause a subject to take”. The intake may be voluntary (free intake) or forced (forced intake). That is, the administration step may be specifically, for example, a step of blending an oligosaccharide and one, two or more tocopherols selected from the group consisting of β-tocopherol, γ-tocopherol and δ-tocopherol in a food, a drink or feed for supplying to a subject and thus causing the subject to freely take the food, the drink or the feed.

The timing of intake (administration) of the composition of the invention (including the first aspect and the second aspect, the same applies below) is not particularly limited and can be appropriately selected according to the condition of the subject of administration.

The intake (administration) dosage of the composition of the invention is appropriately selected based on the age of the subject of intake (administration), the gender, the condition, other conditions and the like.

Regardless of the amount or the period of intake (administration), the medicine can be administered once a day or in multiple divided portions.

The composition of the invention itself may be in the form of a food, a drink, a pharmaceutical product or the like or may be in a form which is contained in a food, a drink, a pharmaceutical product or the like as an additive.

The route of intake (administration) of the composition of the invention may be an oral or parenteral route but is generally an oral route. The parenteral intake (administration) may be rectal administration or the like.

The composition of the invention is preferably an aspect as a food or a drink.

The form and the property of the food or the drink are not particularly restricted as long as the effects of the invention are not impaired, and the food or the drink can be produced by a general method using a material which is generally used for a food or a drink.

An aspect which is added to a food, a drink or a pharmaceutical product before use, namely a so-called additive, is also included in the composition of the invention. An example of the aspect of the composition of the first aspect is an additive which is added to a composition containing an oligosaccharide such as a human milk oligosaccharides. The composition containing a human milk oligosaccharides may be preferably pumped breast milk, a formula or the like. Milk after adding the composition of the invention is expected to be taken by a newborn or an infant.

Here, the “addition” may have the meanings of “mixing”. Moreover, both adding the composition of the invention to a food, a drink or a pharmaceutical product and adding a food, a drink or a pharmaceutical product to the composition of the invention may be included.

The food or the drink is generally orally taken but is not limited thereto, and the food or the drink may be, for example, taken nasally or taken through a gastric fistula or an intestinal fistula. For example, the formula which is the composition of the invention described below or breast milk to which the composition of the invention has been added is expected to be taken by a newborn or an infant through a nasogastric feeding tube or the like.

The food or the drink is not limited regarding the form such as liquid, paste, gel solid or powder. Examples thereof include the following examples: tablet candies; wheat products such as breads, macaroni, spaghetti, noodles, cake mixes, frying flours and bread crumbs; instant foods such as instant noodles, cup noodles, retort-pouched-prepared foods, prepared canned foods, microwave foods, instant soups-stews, instant miso soups-clear Japanese soups, canned soups, freeze-dried foods and other instant foods; processed agricultural products such as canned agricultural products, canned fruits, jams-marmalades, pickles, cooked beans, dried agricultural products and cereals (processed grains); processed fishery products such as canned fishery products, fish hams-sausages, fishery paste products, fishery delicacies and Tsukudani (foods boiled down in sweetened soy sauce); processed livestock products such as canned livestock products-pastes and livestock hams-sausages; milk-dairy products such as processed milk, milk beverages, yogurts, lactic acid bacteria beverages, cheeses, ice creams, creams and other dairy products; oils and fats such as butter, margarine and vegetable oils; basic condiments such as soy sauce, soybean paste, sauces, processed tomato condiments, Mirin (sweet sake for seasoning) and vinegars; compound flavor enhancers-foods such as cooking mixes, curry roux, sauces, dressings, noodle broths, spices and other compound flavor enhancers; frozen foods such as frozen food materials, semi-cooked frozen foods and cooked frozen foods; confectioneries such as caramels, candies, chewing gums, chocolates, cookies, biscuits, cakes, pies, snacks, crackers, Japanese-style confectioneries, rice confectioneries, bean confectioneries, desserts, jellies and other confectioneries; luxury beverages such as carbonated drinks, natural juices, fruit juices, fruit juice-containing soft drinks, fruit flesh drinks, fruit granule-containing fruit juices, vegetable drinks, soy milk, soy milk drinks, coffee drinks, tea drinks, drink powders, concentrated drinks, sport drinks, nutritional drinks, alcohols and other luxury beverages, other commercial foods such as baby foods, Furikake (dry Japanese seasonings) and seasonings for Chazuke (boiled rice with hot tea) and the like; nutritional compositions such as formulas (including powdered milk, liquid milk and the like), liquid foods and supplements; functional foods (foods for specified health uses and foods with nutrient function claims); and the like.

Of these, the first aspect is preferably a nutritional composition, and the second aspect is a nutritional composition (however, breast milk is excluded from the nutritional composition). It is understood that features like “with the proviso that breast milk is excluded” or “breast milk is excluded from the nutritional composition” are optional features which can be omitted.

In the invention, the “nutritional composition” is not particularly limited as an aspect of the food or the drink but is preferably a milk formula, a liquid food, a supplement or the like, more preferably a formula. The subject of intake may be an infant, a young child, a child or an adult but is preferably an infant or a young child.

The composition of the aspect is preferably a nutritional composition, more preferably a formula, particularly preferably a milk formula. The nutritional composition preferably is an infant formula, follow-up formula, or a growing-up formula. An “infant formula” as used herein refers to a formula intended for use by an infant aged 0 to 6 months (i.e. during the first 6 months of life). A “follow up formula” as used herein refers to a formula intended for use by an infant aged 6 to 12 months (i.e. 6 months of age and older, up to and including 12 months). The terms follow-up formula and follow-on formulate are used interchangeably herein. A “growing-up formula” as used herein refers to a formula intended for use by a child aged 12 to 36 months (i.e. a child at the age of 36 months or under the age of 36 months and older than 12 months). The terms “growing-up formula” and “formula for a young child/young children” are used interchangeably herein.

The term “infant” means a child which is 0 to 12 months old (in other words a child at the age of 12 months or under the age of 12 months). The term “young child” means a child which is between 12 to 36 months old (in other words a child at the age of 36 months or under the age of 36 months and older than 12 months). A “young child” is also referred to herein as “toddler”.

The formulas include a powdered formula and a liquid formula. The milk formulas include a powdered milk formula and a liquid milk formula.

The powdered milk formula is defined by the Ministerial Ordinance Concerning Compositional Standards, etc. for Milk and Milk Products as “those obtained by processing raw milk, cow's milk, certified milk or a food produced therefrom as a raw material or using such a material as a major raw material, adding nutrients necessary for infants and young children and processing into powder”.

The liquid milk formula is defined by the Ministerial Ordinance as “those obtained by processing raw milk, cow's milk, certified milk or a food produced therefrom as a raw material or using such a material as a major raw material, adding nutrients necessary for infants and young children and processing into liquid”.

The milk formulas also include those which contain a nutrient component such as proteins, oils and fats, carbohydrates, minerals and vitamins and which are processed into powder or liquid.

The milk formulas further include “a powdered infant formula”, “a liquid infant formula” and “a powdered milk formula for pregnant and parturient women and nursing mothers” of the food for special dietary uses provided by the Health Promotion Act and also include aspects as a powdered infant formula for young children, nutritional powder for adults, nutritional powder for the older adults and the like.

When the composition of the invention is in the form of a supplement, the composition can be formulated into a solid preparation such as powder, granules, tablets and capsules, a liquid preparation such as a solution, a syrup, a suspension and an emulsion or the like. The formulation can be in accordance with the explanations for the component, the carrier and the method for formulation of the pharmaceutical product described below.

An aspect of the food or the drink can be feed. The feed may be pet food, livestock feed, fish farming feed or the like.

The form of the feed is not particularly restricted, and the feed may contain, for example the following materials: grain such as corn, wheat, barley, rye and milo; vegetable oil cake such as soybean oil cake, rapeseed oil cake, coconut oil cake and linseed oil cake; bran such as oat bran, wheat bran, rice bran and defatted rice bran; a food manufacturer's by-product such as corn gluten meal and corn jam meal; animal feed such as fish powder, skim milk powder, whey, yellow grease and tallow; yeast such as torula yeast and brewer's yeast; mineral feed such as tertiary calcium phosphate and calcium carbonate; an oil or a fat; a single amino acid; a saccharide; or the like.

When the composition of the invention is an aspect as a food or a drink (including feed), the composition can be provided-sold as a food or a drink labeled with use for promoting the assimilation of an oligosaccharide by an intestinal bacterium or use for growing a bifidobacterial strain in the intestines.

The “labeling” act includes all the acts for informing a consumer of the use, and all the expressions which can remind of cause to guess the use are the “labeling” acts of the invention, regardless of the purposes of labeling, the contents of labeling, the objects to be labeled, the media and the like.

The “label” is preferably with an expression which allows a consumer to directly recognize the use. Specific examples include an act of transferring an article in which the use is described on a product regarding the food or the drink or packaging of a product, delivering such an article, displaying such an article for transfer or delivery or importing such an article, an act of displaying or distributing an advertisement of a product, a price list or a business document with a description of the use thereon or providing information with such contents with a description of the use by an electromagnetic method (internet or the like) and another act.

The content of the label is preferably a label approved by the administration or the like (for example, a label approved based on a system provided by the administration and provided in the form based on the approval or the like). It is preferable to label with such a content on packaging, a container, a catalogue, a brochure, an advertisement material in a sales site such as POP, other documents or the like.

The “labels” also include labels with health foods, functional foods, enteral nutrition products, food for special dietary uses, food with health claims, foods for specified health uses, foods with nutrient function claims, foods with function claims, quasi-drugs and the like. In particular, the labels are labels approved by the Consumer Affairs Agency, such as labels approved by the systems for foods for specified health uses, foods with nutrient function claims or foods with function claims or by a similar system and the like. Specific examples include a label with foods for specified health uses, a label with qualified foods for specified health uses, a label indicating influence on the structure or the function of a body, a label with reduction of disease risk, a label with a scientifically grounded function and the like. More specifically, typical examples include labels with food for specified health uses (especially labels with health uses) provided by the Cabinet Office Ordinance on Labeling Permission for Special Dietary Uses under the Health Promotion Act (Cabinet Office Ordinance No. 57 on Aug. 31, 2009) and similar labels.

The labels are, for example, labels with “for efficient use of prebiotics”, “for helping the functions of oligosaccharides which help the health of babies”, “for health of the gut condition of infants and young children”, “those who wish to increase bifidobacteria”, “for increasing bifidobacteria which help the health of babies”, “improving the enteric microbiota”, “improving the intestinal microbiota” and the like.

When the composition of the invention is in the form of a pharmaceutical product, the route of administration thereof may be an oral or parenteral route but is preferably an oral route. The parenteral intake (administration) may be rectal administration or the like.

Regarding the form of the pharmaceutical product, the composition can be appropriately formulated into a desired dosage form depending on the administration method. For example, in the case of oral administration, the composition can be formulated into a solid preparation such as powder, granules, tablets and capsules, a liquid preparation such as a solution, a syrup, a suspension and an emulsion or the like. In the case of parenteral administration, the composition can be formulated into a suppository, ointment, an injection or the like.

For the formulation, a component which is generally used for formulation such as excipients, pH-adjusting agents, colorants and corrigents can be used. Another medicinal component, a prebiotic material which is known or will be found in the future or the like can also be used in combination. As known prebiotics, inulin, gluconic acid, polydextrose and the like are preferable as those contained in the composition of the invention.

In addition, the formulation can be conducted by an appropriate known method depending on the dosage form. The formulation may also be conducted by appropriately blending a carrier for formulation.

Examples of the excipients include: saccharide derivatives such as lactose, sucrose, glucose, mannitol and sorbitol; starch derivatives such as cornstarch, potato starch, α-starch, dextrin and carboxymethyl starch; cellulose derivatives such as crystalline cellulose, hydroxypropyl cellulose, hydroxypropyl methylcellulose, carboxymethylcellulose and carboxymethyl cellulose calcium; gum arabic; dextran; pullulan; silicate derivatives such as light silicic anhydride, synthetic aluminum silicate and magnesium aluminometasilicate; phosphate derivatives such as calcium phosphate; carbonate derivatives such as calcium carbonate; sulfate derivatives such as calcium sulfate; and the like.

Examples of binders include, in addition to the excipients, gelatin, polyvinylpyrrolidone, macrogol and the like.

Examples of disintegrating agents include, in addition to the excipients, chemically modified starch or cellulose derivatives such as croscarmellose sodium, sodium carboxymethyl starch and cross-linked polyvinylpyrrolidone and the like.

Examples of lubricants include: talc; stearic acid; metal stearates such as calcium stearate and magnesium stearate; colloidal silica; waxes such as Veegum and spermaceti wax; boric acid; glycols; carboxylic acids such as fumaric acid and adipic acid; sodium carboxylates such as sodium benzoate; sulfates such as sodium sulfate; leucine; lauryl sulfates such as sodium lauryl sulfate and magnesium lauryl sulfate; silicic acid such as silicic anhydride and silicic acid hydrate; starch derivatives; and the like.

Examples of stabilizers include: paraoxybenzoate esters such as methylparaben and propylparaben; alcohols such as chlorobutanol, benzyl alcohol and phenylethyl alcohol; benzalkonium chloride; acetic anhydride; sorbic acid; and the like.

Examples of flavoring agents include sweeteners, acidulants, aromas and the like.

In this regard, the carriers used in the case of a liquid preparation for oral administration include solvents such as water and the like.

The timing for taking the pharmaceutical product of the invention is not particularly limited, and examples include before a meal, after a meal, between meals, before bedtime and the like.

As used herein, the terms “comprising”, “including”, “having” or grammatical variants thereof are to be taken as specifying the stated features, integers, steps or components but do not preclude the addition of one or more additional features, integers, steps, components or groups thereof. The terms “comprising”/“including”/“having” encompass the terms “consisting of” and “consisting essentially of”. Thus, whenever the terms “comprising”/“including”/“having” are used herein, they can be replaced by “consisting essentially of” or, preferably, by “consisting of”. The terms “comprising” and “containing” are used interchangeably herein.

The terms “comprising”/“including”/“having” mean that any further component (or likewise features, integers, steps and the like) can be present.

The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed product, composition, device or method and the like.

Thus, the term “consisting essentially of” means that specific further components (or likewise features, integers, steps and the like) can be present, namely those not materially affecting the essential characteristics of the product, composition, device or method. In other words, the term “consisting essentially of” (which can be interchangeably used herein with the term “comprising substantially”), allows the presence of other components in the product, composition, device or method in addition to the mandatory components (or likewise features, integers, steps and the like), provided that the essential characteristics of the product, composition, device or method are not materially affected by the presence of other components. As used herein, “a” or “an” may mean one or more.

### EXAMPLES

The invention is explained further specifically below using Examples, but the invention is not limited to the Examples.

### <Test Example 1> Evaluation of Assimilation Promotion Action of Human Milk Oligosaccharides by Fecal Culture

### (1) Pretreatment of Samples

Fecal samples were obtained from two healthy infants (feces A from a 1.5-month-old infant and feces B from a 7-month-old infant). The samples were collected immediately after the defecation and immediately moved to 10° C. or lower under an anaerobic condition. The collected feces were diluted with 10 times the amount of physiological saline within eight hours of the defecation and stored at −80° C. until use.

### (2) Fecal Culture

To imitate the environment of the large intestine, a culture experiment was conducted by the following procedures. Bio Jr. 8 100 mLx8 culture apparatus (BJR-25NA1S-8M manufactured by Biott Corporation) was used, and the operations were according to the instructions of the device. As the medium, 100 mL of YCFA medium having the composition shown in Table 2 was used. The medium components except for the tocopherols and the oligosaccharides were dissolved in purified water and sterilized in an autoclave. The tocopherols and the oligosaccharides which were dissolved in dimethyl sulfoxide (DMSO, FUJIFILM Wako Pure Chemical Corporation) and in purified water, respectively, and which were then sterilized with a filter were added to the medium after the autoclave treatment. The culture temperature was 37° C., and the anaerobic condition was maintained during the culture period by blowing filter-sterilized CO₂ into the culture vessels. After adjusting the pH to 7.0, 100 μL (10 mg in terms of feces) of the fecal sample (feces B), which was diluted with physiological saline, was added, and anaerobic culture was started. To imitate the pH in the large intestine of an infant, the pH was controlled by neutralizing with 1M Na₂CO₃ when the pH became lower than 5.5 during the culture period. The media were collected 7, 10 and 24 hours after starting the culture and centrifuged at 8000 rpm for 3 min at 4° C., and the supernatants and the precipitates were collected.

TABLE 2
Medium Composition

| | | | | | | | Example 2 |
|---|---|---|---|---|---|---|---|
| | | | Comparative | Example 1-1 | Example 1-2 | Comparative | 6′-SL + |
| | | | Example 1 | Example 8 | 2′-FL + | Example 2 | Fructo- |
| | | Unit/ | Comparative | 2′-FL + | LNT + | 6′-SL + | oligosaccharide + |
| | | 100 | Example 8 | Tocopherol | Tocopherol | Fructo- | Tocopherol |
| | Reagent Source | mL | 2′-FL | Pattern 3 | Pattern 3 | oligosaccharide | Pattern 3 |
| Casitone | Becton, Dickinson and Company | g | 1.0 | 1.0 | 1.0 | 1.0 | 1.0 |
| Yeast extract | Becton, Dickinson and Company | g | 0.25 | 0.25 | 0.25 | 0.25 | 0.25 |
| NaHCO₃ | Kokusan Chemical Co., Ltd. | g | 0.4 | 0.4 | 0.4 | 0.4 | 0.4 |
| KH₂PO₄ | Kokusan Chemical Co., Ltd. | g | 0.045 | 0.045 | 0.045 | 0.045 | 0.045 |
| NaCl | Kokusan Chemical Co., Ltd. | g | 0.09 | 0.09 | 0.09 | 0.09 | 0.09 |
| (NH₄) ₂SO₄ | Kokusan Chemical Co., Ltd. | g | 0.09 | 0.09 | 0.09 | 0.09 | 0.09 |
| MgSO₄•7H₂O | Kokusan Chemical Co., Ltd. | g | 0.045 | 0.045 | 0.045 | 0.045 | 0.045 |
| CaCl₂ | Kokusan Chemical Co., Ltd. | g | 0.09 | 0.09 | 0.09 | 0.09 | 0.09 |
| L-Cysteine-HCl | Kanto Chemical Co., Inc. | g | 0.145 | 0.145 | 0.145 | 0.145 | 0.145 |
| Resazurin | FUJIFILM Wako Pure Chemical | mg | 0.1 | 0.1 | 0.1 | 0.1 | 0.1 |
| Hemin | FUJIFILM Wako Pure Chemical | mg | 1.0 | 1.0 | 1.0 | 1.0 | 1.0 |
| Biotin | Nacalai Tesque, Inc. | μg | 1.0 | 1.0 | 1.0 | 1.0 | 1.0 |
| Vitamin B12 | Nacalai Tesque, Inc. | μg | 1.0 | 1.0 | 1.0 | 1.0 | 1.0 |
| p-Aminobenzoic | Nacalai Tesque, Inc. | μg | 3.0 | 3.0 | 3.0 | 3.0 | 3.0 |
| Acid | | | | | | | |
| Folic Acid | Nacalai Tesque, Inc. | μg | 5.0 | 5.0 | 5.0 | 5.0 | 5.0 |
| Pyridoxamine | Nacalai Tesque, Inc. | μg | 15.0 | 15.0 | 15.0 | 15.0 | 15.0 |
| Dihydrochloride | | | | | | | |
| Vitamin B1 | Nacalai Tesque, Inc. | μg | 5.0 | 5.0 | 5.0 | 5.0 | 5.0 |
| Riboflavin | FUJIFILM Wako Pure Chemical | μg | 5.0 | 5.0 | 5.0 | 5.0 | 5.0 |

TABLE 3
Medium Composition (Continued)

| | | | | | | | Example 2 |
|---|---|---|---|---|---|---|---|
| | | | Comparative | Example 1-1 | Example 1-2 | Comparative | 6′-SL + |
| | | | Example 1 | Example 8 | 2′-FL + | Example 2 | Fructo- |
| | | Unit/ | Comparative | 2′-FL + | LNT + | 6′-SL + | oligosaccharide + |
| | | 100 | Example 8 | Tocopherol | Tocopherol | Fructo- | Tocopherol |
| | Reagent Source | mL | 2′-FL | Pattern 3 | Pattern 3 | oligosaccharide | Pattern 3 |
| Acetic acid | FUJIFILM Wako Pure Chemical | mL | 0.2 | 0.2 | 0.2 | 0.2 | 0.2 |
| Propionic acid | FUJIFILM Wako Pure Chemical | mL | 0.07 | 0.07 | 0.07 | 0.07 | 0.07 |
| n-Valeric acid | Kanto Chemical Co., Inc. | mL | 0.012 | 0.012 | 0.012 | 0.012 | 0.012 |
| Iso-Valeric acid | Kanto Chemical Co., Inc. | mL | 0.012 | 0.012 | 0.012 | 0.012 | 0.012 |
| Iso-butyric acid | Kanto Chemical Co., Inc. | mL | 0.012 | 0.012 | 0.012 | 0.012 | 0.012 |
| β-Tocopherol | FUJIFILM Wako Pure Chemical | mg | — | 0.57 | 0.57 | — | 0.57 |
| γ-Tocopherol | FUJIFILM Wako Pure Chemical | mg | — | 22.5 | 22.5 | — | 22.5 |
| δ-Tocopherol | FUJIFILM Wako Pure Chemical | mg | — | 5.7 | 5.7 | — | 5.7 |
| 2′- | Jennewein Biotechnologie GmbH | g | 0.2 | 0.2 | 0.2 | — | — |
| Fucosyllactose | | | | | | | |
| (2′-FL) | | | | | | | |
| 6′-Sialyllactose | Jennewein Biotechnologie GmbH | g | — | — | — | 0.2 | 0.2 |
| (6′-SL) | | | | | | | |
| Lacto-N-tetraose | Jennewein Biotechnologie GmbH | g | — | — | 0.2 | | |
| (LNT) | | | | | | | |
| Fructooligosaccharide | Teijin Limited/Fuji Nibon | g | — | — | — | 0.8 | 0.8 |
| | Seito Corporation | | | | | | |
| Galactooligosaceharide | FUJIFILM Wako Pure Chemical | g | — | — | — | — | — |
| | Corporation | | | | | | |
| γ/δ | | | | 3.9 | 3.9 | | 3.9 |
| δ/β | | | | 10 | 10 | | 10 |

TABLE 4
Medium Composition (Continued)

| | | | | Example 9 | | Comparative | Comparative |
|---|---|---|---|---|---|---|---|
| | | Unit/ | Comparative | 6′-SL + | Comparative | Example 10-1 | Example 10-2 |
| | | 100 | Example 9 | Tocopherol | Example 10-0 | Tocopherol | Tocopherol |
| | Reagent Source | mL | 6′-SL | Pattern 3 | Control | Pattern 1 | Pattern 4 |
| Casitonie | Becton, Dickinson and | g | 1.0 | 1.0 | 1.0 | 1.0 | 1.0 |
| | Company | | | | | | |
| Yeast extract | Becton, Dickinson and | g | 0.25 | 0.25 | 0.25 | 0.25 | 0.25 |
| | Company | | | | | | |
| NaHCO₃ | Kokusan Chemical Co., Ltd. | g | 0.4 | 0.4 | 0.4 | 0.4 | 0.4 |
| KH₂PO₄ | Kokusan Chemical Co., Ltd. | g | 0.045 | 0.045 | 0.045 | 0.045 | .045 |
| NaCl | Kokosan Chemical Co., | g | 0.09 | 0.09 | 0.09 | 0.09 | 0.09 |
| (NH₄) ₂SO₄ | Kokusan Chemical Co., Ltd. | g | 0.09 | 0.09 | 0.09 | 0.09 | 0.09 |
| MgSO₄•7H₂O | Kokusan Chemical Co., Ltd | g | 0.045 | 0.045 | 0.045 | 0.045 | 0.045 |
| CaCl₂ | Kokusan Chemical Co., Ltd. | g | 0.09 | 0.09 | 0.09 | 0.09 | 0.09 |
| L-Cysteine-HCl | Kanto Chemical Co., Inc. | g | 0.145 | 0.145 | 0.145 | 0.145 | 0.145 |
| Resazurin | FUJIFILM Wako Pure Chemical | mg | 0.1 | 0.1 | 0.1 | 0.1 | 0.1 |
| Hemin | FUJIFILM Wako Pure Chemical | mg | 1.0 | 1.0 | 1.0 | 1.0 | 1.0 |
| Biotin | Nacalai Tesque, Inc. | μg | 1.0 | 1.0 | 1.0 | 1.0 | 1.0 |
| Vitamin B12 | Nacalai Tesque, Inc. | μg | 1.0 | 1.0 | 1.0 | 1.0 | 1.0 |
| p-Aminobenzoic Acid | Nacalai Tesque, Inc. | μg | 3.0 | 3.0 | 3.0 | 3.0 | 3.0 |
| Folic Acid | Nacalai Tesque, Inc. | μg | 5.0 | 5.0 | 5.0 | 5.0 | 5.0 |
| Pyridoxamine | Nacalai Tesque, Inc. | μg | 15.0 | 15.0 | 15.0 | 15.0 | 15.0 |
| Dihydrochloride | | | | | | | |
| Vitamin B1 | Nacalai Tesque, Inc. | μg | 5.0 | 5.0 | 5.0 | 5.0 | 5.0 |
| Riboflavin | FUJIFILM Wako Pure Chemical | μg | 5.0 | 5.0 | 5.0 | 5.0 | 5.0 |
| Acetic acid | FUJIFILM Wako Pure Chemical | μg | 0.2 | 0.2 | 0.2 | 0.2 | 0.2 |
| Propionic acid | FUJIFILM Wako Pure Chemical | mL | 0.07 | 0.07 | 0.07 | 0.07 | 0.07 |
| n-Valeric acid | Kanto Chemical Co., Inc. | mL | 0.012 | 0.012 | 0.012 | 0,012 | 0.012 |
| Iso-Valeric acid | Kanto Chemical Co., Inc. | mL | 0.012 | 0.012 | 0.012 | 0.012 | 0.018 |
| Iso-butyric acid | Kanto Chemical Co., Ino | mL | 0.012 | 0.012 | 0.012 | 0.012 | 0.012 |

TABLE 5
Medium Composition (Continued)

| | | | | | | Comparative | |
|---|---|---|---|---|---|---|---|
| | | | | Example 9 | Comparative | Example | Comparative |
| | | Unit/ | Comparative | 6′-SL + | Example | 10-1 | Example 10-2 |
| | | 100 | Example 9 | Tocopherol | 10-0 | Tocopherol | Tocopherol |
| | Reagent Source | mL | 6′-SL | Pattern 3 | Control | Pattern 1 | Pattern 4 |
| β-Tocopherol | FUJIFILM Wako Pure Chemical | mg | — | 0.57 | — | 0.57 | 0.57 |
| | Corporation | | | | | | |
| γ-Tocopherol | FUJIFILM Wako Pure Chemical | mg | — | 22.5 | — | 14.1 | 25.2 |
| | Corporation | | | | | | |
| δ-Tocopherol | FUJIFILM Wako Pure Chemical | mg | — | 5.7 | — | 14.1 | 2.9 |
| | Corporation | | | | | | |
| 2′-Fucosyllactose (2′-FL) | Vennewein Biotechnologie GmbH | g | — | — | — | — | — |
| 6′-Sialyllactose (6′-SL) | Jennewein Biotechnologie GmbH | g | 0.2 | 0.2 | — | — | — |
| Lacto-N-tetraose (INT) | Jennewein Biotechnologie GmbH | g | — | — | — | — | — |
| Fructooligosaccharide | Teijin Limited/Fuji Nihon Seito | g | — | — | — | — | — |
| | Corporation | | | | | | |
| Galactooligosaccharide | FUJIFILM Wako Pure Chemical | g | — | — | — | — | — |
| | Corporation | | | | | | |
| γ/δ | | | | 3.9 | | 1 | 8.7 |
| δ/β | | | | 10 | | 24.6 | 5.1 |

TABLE 6
Medium Composition

| | | | Comparative | Example 3-1 | Example 3-2 | Example 3-3 | Example 3-4 |
|---|---|---|---|---|---|---|---|
| | | | Example 3 | Example 11-1 | Example 11-2 | Example 11-3 | Example 11-4 |
| | | | Comparative | Galacto- | Galacto- | Galacto- | Galacto- |
| | | Unit/ | Example 11 | oligosaccharide + | oligosaccharide + | oligosaccharide + | oligosaccharide + |
| | Reagent | 100 | Galacto- | Tocopherol | Tocopherol | Tocopherol | Tocopherol |
| | Source | mL | oligosaccharide | Pattern 1 | Pattern 2 | Pattern 3 | Pattern 4 |
| Casitone | Becton, Dickinson | g | 1.0 | 1.0 | 1.0 | 1.0 | 1.0 |
| | and Company | | | | | | |
| Yeast extract | Becton, Dickinson | g | 0.25 | 0.25 | 0.25 | 0.25 | 0.25 |
| | and Company | | | | | | |
| NaHCO₃ | Kokusan Chemical Co., Ltd. | g | 0.4 | 0.4 | 0.4 | 0.4 | 0.4 |
| KH [symbol] PO₄ | Kokusan Chemical Co., Ltd. | g | 0.045 | 0.045 | 0.045 | 0.045 | 0.045 |
| NaCl | Kokusan: Chemical Co., Ltd. | g | 0.09 | 0.09 | 0.09 | 0.09 | 0.09 |
| (NH [symbol] ) ₇SO₄ | Kokusan Chemical Co., Ltd. | g | 0.09 | 0.09 | 0.09 | 0.09 | 0.09 |
| MgSO₄•₇H₂O | Kokusan Chemical Co., Ltd. | g | 0.045 | 0.045 | 0.045 | 0.045 | 0.045 |
| CaCl [symbol] | Kokusan Chemical Co., Ltd. | g | 0.09 | 0.09 | 0.09 | 0.09 | 0.09 |
| L-Cysteine-HCl | Kanto Chemical Co., Inc. | g | 0.145 | 0.145 | 0.145 | 0.145 | 0.145 |
| Resazurin | FUJIFILM Wako | mg | 0.1 | 0.1 | 0.1 | 0.1 | 0.1 |
| | Pure Chemical | | | | | | |
| Hemin | FUJIFILM Wako | mg | 1.0 | 1.0 | 1.0 | 1.0 | 1.0 |
| | Pure Chemical | | | | | | |
| Biotin | Nacalai Tesque, Inc. | μg | 1.0 | 1.0 | 1.0 | 1.0 | 1.0 |
| Vitamin B12 | Nacalai Tesque, Inc. | μg | 1.0 | 1.0 | 1.0 | 1.0 | 1.0 |
| p-Aminobenzoic | Nacalai Tesque, Inc. | μg | 3.0 | 3.0 | 3.0 | 3.0 | 3.0 |
| Acid | | | | | | | |
| Folic Acid | Nacalai Tesque, Inc. | μg | 5.0 | 5.0 | 3.0 | 5.0 | 5.0 |
| Pyridoxamine | Nacalai Tesque, Inc. | μg | 15.0 | 15.0 | 15.0 | 15.0 | 15.0 |
| Dihydrochloride | | | | | | | |
| Vitamin B1 | Nacalai Tesque, Inc. | μg | 5.0 | 5.0 | 5.0 | 5.0 | 5.0 |
| Riboflavin | FUJIFILM Wako | μg | 5.0 | 5.0 | 5.0 | 5.0 | 5.0 |
| | Pure Chemical | | | | | | |
| Acetic acid | FUJIFILM Wako | mL | 0.2 | 0.2 | 0.2 | 0.2 | 0.2 |
| | Pure Chemical | | | | | | |
| Propionic acid | FUJIFILM Wako | mL | 0.07 | 0.07 | 0.07 | 0.07 | 0.07 |
| | Pure Chemical | | | | | | |

[symbol]

indicates data missing or illegible when filed

TABLE 7
Medium Composition (Continued)

| | | | Comparative | Example 3-1 | Example 3-2 | Example 3-3 | Example 3-4 |
|---|---|---|---|---|---|---|---|
| | | | Example 3 | Example 11-1 | Example 11-2 | Example 11-3 | Example 11-4 |
| | | | Comparative | Galacto- | Galacto- | Galacto- | Galacto- |
| | | Unit/ | Example 11 | oligosaccharide + | oligosaccharide + | oligosaccharide + | oligosaccharide + |
| | | 100 | Galacto- | Tocopherol | Tocopherol | Tocopherol | Tocopherol |
| | Reagent Source | mL | oligosaccharlide | Pattern 1 | Pattern 2 | Pattern 3 | Pattern 4 |
| n-Valeric acid | Kanto | mL | 0.012 | 0.012 | 0,012 | 0.012 | 0.012 |
| | Chemical Co., Inc. | | | | | | |
| Iso-Valeric acid | Kanto | mL | 0.012 | 0.012 | 0.012 | 0.012 | 0.012 |
| | Chemical Co., Inc. | | | | | | |
| Iso-butyric acid | Kanto | mL | 0.012 | 0.012 | 0.012 | 0.012 | 0.012 |
| | Chemical Co., Inc. | | | | | | |
| β-Tocopherol | FUJIFILM Wako | mg | — | 0.57 | 0.57 | 0.57 | 0.57 |
| | Pure Chemical | | | | | | |
| | Corporation | | | | | | |
| γ-Tocopherol | FUJIFILM Wako | mg | — | 14.2 | 18.9 | 22.5 | 25.2 |
| | Pure Chemical | | | | | | |
| | Corporation | | | | | | |
| δ-Tocopherol | FUJIFILM Wako | mg | — | 14.1 | 9.2 | 5.7 | 2.9 |
| | Pure Chemical | | | | | | |
| | Corporation | | | | | | |
| 2′-Fucosyllactose | Tennenein | g | — | — | — | — | — |
| (2′-FL) | Biotechnologie GmbH | | | | | | |
| 6′-Sialyilactose | Jennewein | g | — | — | — | — | — |
| (6′-SL) | Biotechnologie GmbH | | | | | | |
| Lacto-N-tetraose | Jennewein | g | — | — | — | — | — |
| (INT) | Biotechnologie GmbH | | | | | | |
| Fructooligosaccharide | Teijin Limited/ | g | — | — | — | — | — |
| | Fuji Nihon Seito | | | | | | |
| | Corporation | | | | | | |
| Galactooligosaccharid | FUJIFILM Wako | g | 0.8 | 0.8 | 0.8 | 0.8 | 0.8 |
| | Pure Chemical | | | | | | |
| | Corporation | | | | | | |
| γ/δ | | | | 1 | 2.1 | 3.9 | 8.7 |
| δ/β | | | | 24.6 | 16.1 | 10 | 5.1 |

### (3) Analysis of Oligosaccharide Amounts in Culture Supernatants

An equivalent amount of acetonitrile was added to the collected supernatants which were diluted with four times the amount of water, and the mixtures were centrifuged (21500 g, 15 minutes, 4° C.), filtered through a filter, then diluted 100 times with 50% acetonitrile and subjected to analysis of oligosaccharides by LC-MS.

For the UHPLC separation, Vanquish Flex UHPLC (Thermo Fisher Scientific, Waltham, MA, U.S.A.) system and ACQUITY UPLC Glycoprotein BEH amide column (1.7 μm, 2.1 mm×150 mm, Waters, Milford, MA, USA) were used. The gradient eluents were A buffer (10 mmol/L ammonium formate, 0.1% formic acid) and B buffer (0.1% formic acid/acetonitrile), and the flow rate was 0.35 ml/min. The column temperature was set at 35° C. The gradient elution of 13.5 min included elution with B buffer at 75% to 60% over 7.5 min and at 60% to 20% over 1 min, holding at 20% for 1 min, then elution at 20% to 75% over 0.1 min and final holding at 75% for 4.4 min. The mass analysis was conducted using a heated electrospray ion source, Orbitrap mass spectrometry Q Exactive Focus (Thermo Fisher Scientific) in the parallel reaction monitoring (PRM) mode. The data analysis was carried out using TraceFinder software (Thermo Fisher Scientific).

### (4) Results

The residual rates of the oligosaccharides in the culture supernatants are shown in Table 3, where the values before the culture are 100%.

Although 2′-fucosyllactose remained in the supernatant without being assimilated after seven hours of culture (Comparative Example 1), the residual rate decreased to about 70% when three kinds of tocopherol, namely β-, γ- and δ-tocopherols, were added (Example 1-1). Moreover, when lacto-N-tetraose (LNT) was added in addition to the three kinds of tocopherol, the residual rate after 10 hours of culture decreased to around 20% of the value without the addition of LNT (Example 1-2).

Although 6′-sialyllactose remained in the supernatant without being assimilated even after 24 hours of culture in the presence of fructooligosaccharide (Comparative Example 2), the residual rate decreased to around 20% when three kinds of tocopherol, namely β-, γ- and δ-tocopherols, were added (Example 2).

Although the residual rate of galactooligosaccharide after seven hours of culture was 70% (Comparative Example 3), the residual rate of galactooligosaccharide decreased to about 50% in each case when three kinds of tocopherol, namely β-, γ- and δ-tocopherols, were added at different ratios (Examples 3-1 to 3-4).

From the results, it was shown that the three kinds of tocopherol, namely β-, γ- and δ-tocopherols, promote the assimilation of oligosaccharides such as human milk oligosaccharides.

TABLE 8
Table 3-1: 2′-FL Residual Rate (mass %)

| Group | | Before | After 7-hr |
|---|---|---|---|
| Structure | | Culture | Culture |
| Comparative | 2′-FL | 100 | 114 |
| Example 1: | | | |
| Example 1-1: | 2′-FL + Tocopherol Pattern 3 | 100 | 73 |

TABLE 9
Table 3-2: 2′-FL Residual Rate (mass %)

| Group | | Before | After 10-hr |
|---|---|---|---|
| Structure | | Culture | Culture |
| Example 1-1: | 2′-FL + Tocopherol Pattern 3 | 100 | 74 |
| Example 1-2: | 2′-FL + LNT + Tocopherol | 100 | 16 |
| | Pattern 3 | | |

TABLE 10
Table 3-3: 6′-SL Residual Rate (mass %)

| Group | | Before | After 24-hr |
|---|---|---|---|
| Structure | | Culture | Culture |
| Comparative | 6′-SL + Fructooligosaccharide | 100 | 97 |
| Example 2: | 6′- | | |
| Example 2: | SL + Fructooligosaccharide + | 100 | 19 |
| | Tocopherol Pattern 3 | | |

TABLE 11
Table 3-4: Galactooligosaccharide Residual Rate (mass %)

| | | | | | After |
|---|---|---|---|---|---|
| Group | | | | Before | 7-hr |
| Structure | | γ/δ | δ/β | Culture | Culture |
| Comparative | Galactooligosaccharide | | | 100 | 70 |
| Example 3: | | | | | |
| Example 3-1: | Galactooligosaccharide + | 1 | 24.6 | 100 | 47 |
| | Tocopherol Pattern 1 | | | | |
| Example 3-2: | Galactooligosaccharide + | 2.1 | 16.1 | 100 | 47 |
| | Tocopherol Pattern 2 | | | | |
| Example 3-3: | Galactooligosaccharide + | 3.9 | 10 | 100 | 52 |
| | Tocopherol Pattern 3 | | | | |
| Example 3-4: | Galactooligosaccharide + | 8.7 | 5.1 | 100 | 50 |
| | Tocopherol Pattern 4 | | | | |

### <Test Example 2> Evaluation of Bifidobacterial Strain Growth Promotion Action by Single Culture of Bacterial Strains

### (1) Single Culture

Bifidobacterium infantis M-63, Bifidobacterium infantis MCC2042 or Bifidobacterium bifidum MCC2030 was cultured in MRS medium at 37° C. for 16 hours, and preculture solutions were produced.

The components shown in Table 4 (except for cysteine, the tocopherols and the oligosaccharides) were dissolved in purified water to produce a medium of 2× concentration, and the medium was sterilized in an autoclave at 121° C. for 15 minutes after adjusting the pH to 6.5. Then, filter-sterilized cysteine was added. The tocopherols and the oligosaccharides were dissolved in dimethyl sulfoxide (FUJIFILM Wako Pure Chemical) and in purified water, respectively, sterilized with a filter and added to the medium, and then the concentrations were adjusted with sterilized purified water to achieve the final concentrations shown in Table 4. The prepared media were added to a 96-well plate each in a volume of 200 μL (n=6 per group), and subsequently, the preculture solutions of Bifidobacterium infantis M-63, Bifidobacterium infantis MCC2042 and Bifidobacterium bifidum MCC2030 were aseptically added under an anaerobic condition each at 1% of the medium volume. Culture was conducted at 37° C. for 16 hours in an anaerobic chamber (Baker Ruskinn), and the turbidities (OD₆₀₀) were measured.

TABLE 12
Table 4-1: Medium Composition

| | | | | Comparative | Comparative | Comparative | Comparative |
|---|---|---|---|---|---|---|---|
| | | | Comparative | Example 4-1 | Example 4-2 | Example 4-3 | Example 4-4 |
| | | Unit/ | Example 4-0 | Tocopherol | Tocopherol | Tocopherol | Tocopherol |
| | Reagent Source | 100 mL | Control | Pattern 1 | Pattern 2 | Pattern 3 | Pattern 4 |
| Peptone | Becton, Dickinson and Company | g | 1 | 1 | 1 | 1 | 1 |
| Beef extract | Becton, Dickinson and Company | g | 1 | 1 | 1 | 1 | 1 |
| Yeast extract | Becton, Dickinson and Company | g | 0.5 | 0.5 | 0.5 | 0.5 | 0.5 |
| Tween80 | nacalai tesque | g | 0.1 | 0.1 | 0.1 | 0.1 | 0.1 |
| K₂HPO₄ | Kokusan Chemical | g | 0.2 | 0.2 | 0.2 | 0.2 | 0.2 |
| Na Acetate | FUJIFILM Wako Pure Chemical | g | 0.5 | 0.5 | 0.5 | 0.5 | 0.5 |
| | Corporation | | | | | | |
| Diammonium | FUJIFILM Wako Pure Chemical | g | 0.2 | 0.2 | 0.2 | 0.2 | 0.2 |
| Hydrogen | Corporation | | | | | | |
| Citrate | | | | | | | |
| MgSO₄•7H₂O | FUJIFILM Wako Pure Chemical | mg | 20 | 20 | 20 | 20 | 20 |
| | Corporation | | | | | | |
| MnSO₄•5H₂O | FUJIFILM Wako Pure Chemical | mg | 5 | 5 | 5 | 5 | 5 |
| | Corporation | | | | | | |
| L-Cysteine- | Kanto Chemical Co., Inc. | mg | 50 | 50 | 50 | 50 | 5.0 |
| HCl | | | | | | | |
| β-Tocopherol | FUJIFILM Wako Pure Chemical | mg | 0 | 0.57 | 0.57 | 0.57 | 0.57 |
| | Corporation | | | | | | |
| γ-Tocopherol | FUJIFILM Wako Pure Chemical | mg | 0 | 14.1 | 18.9 | 22.5 | 25.2 |
| | Corporation | | | | | | |
| δ-Tocopherol | FUJIFILM Wako Pure Chemical | mg | 0 | 14.1 | 9.2 | 5.7 | 2.9 |
| | Corporation | | | | | | |
| γ/δ | | | | 1 | 2.1 | 3.9 | 8.7 |
| δ/β | | | | 24.6 | 16.1 | 10 | 5.1 |

TABLE 13
Table 4-2: Medium Composition

| | | | | Example 5-1 | Example 5-2 | Example 5-3 | Example 5-4 |
|---|---|---|---|---|---|---|---|
| | | | Comparative | 2′-FL + | 2′-FL + | 2′-FL + | 2′-FL + |
| | | Unit/ | Example 5 | Tocopherol | Tocopherol | Tocopherol | Tocopherol |
| | Reagent Source | 100 mL | 2′-FL | Pattern 1 | Pattern 2 | Pattern 3 | Pattern 4 |
| Peptone | Becton, Dickinson and Company | g | 1 | 1 | 1 | 1 | 1 |
| Beef extract | Becton, Dickinson and Company | g | 1 | 1 | 1 | 1 | 1 |
| Yeast extract | Becton, Dickinson and Company | g | 0.5 | 0.5 | 0.5 | 0.5 | 0.5 |
| Tween80 | nacalai tesque | g | 0.1 | 0.1 | 0.1 | 0.1 | 0.1 |
| K₂HPO₄ | Kokusan Chemical | g | 0.2 | 0.2 | 0.2 | 0.2 | 0.2 |
| Na Acetate | FUJIFILM Wako Pure Chemical | g | 0.5 | 0.5 | 0.5 | 0.5 | 0.5 |
| | Corporation | | | | | | |
| Diammonium | FUJIFILM Wako Pure Chemical | g | 0.2 | 0.2 | 0.2 | 0.2 | 0.2 |
| Hydrogen | Corporation | | | | | | |
| Citrate | | | | | | | |
| MgSO₄•7H₂O | FUJIFILM Wako Pure Chemical | mg | 20 | 20 | 20 | 20 | 20 |
| | Corporation | | | | | | |
| MnSO₄•5H₂O | FUJIFILM Wako Pure Chemical | mg | 5 | 5 | 5 | 5 | 5 |
| | Corporation | | | | | | |
| L-Cysteine- | Kanto Chemical Co., Inc. | mg | 50 | 50 | 50 | 50 | 50 |
| HCl | | | | | | | |
| Fucosyllactose | Biosynth Carbosynth | g | 0.2 | 0.2 | 0.2 | 0.2 | 0.2 |
| (3′-FL) | | | | | | | |
| β-Tocopherol | FUJIFILM Wako Pure Chemical | mg | 0 | 0.57 | 0.57 | 0.57 | 0.57 |
| | Corporation | | | | | | |
| γ-Tocopherol | FUJIFILM Wako Pure Chemical | mg | 0 | 14.1 | 18.9 | 22.5 | 25.2 |
| | Corporation | | | | | | |
| δ-Tocopherol | FUJIFILM Wako Pure Chemical | mg | 0 | 14.1 | 9.2 | 5.7 | 2.9 |
| | Corporation | | | | | | |
| γ/δ | | | | 1 | 2.1 | 3.9 | 8.7 |
| δ/β | | | | 24.6 | 16.1 | 10 | 5.1 |

TABLE 14
Table 4-3: Medium Composition

| | | | | Example 6-1 | Example 6-2 | Example 6-3 | Example 6-4 |
|---|---|---|---|---|---|---|---|
| | | | Comparative | 6′-SL + | 6′-SL + | 6′-SL + | 6′-SL + |
| | | Unit/ | Example 6 | Tocopherol | Tocopherol | Tocopherol | Tocopherol |
| | Reagent Source | 100 mL | 6′-SL | Pattern 1 | Pattern 2 | Pattern 3 | Pattern 4 |
| Peptone | Becton, Dickinson and Company | g | 1 | 1 | 1 | 1 | 1 |
| Beef extract | Becton, Dickinson and Company | g | 1 | 1 | 1 | 1 | 1 |
| Yeast extract | Becton, Dickinson and Company | g | 0.5 | 0.5 | 0.5 | 0.5 | 0.5 |
| Tween80 | nacalai tesque | g | 0.1 | 0.1 | 0.1 | 0.1 | 0.1 |
| K₂HPO₄ | Kokusan Chemical | g | 0.2 | 0.2 | 0.2 | 0.2 | 0.2 |
| Na Acetate | FUJIFILM Wako Pure Chemical | g | 0.5 | 0.5 | 0.5 | 0.5 | 0.5 |
| | Corporation | | | | | | |
| Diammonium | FUJIFILM Wako Pure Chemical | g | 0.2 | 0.5 | 0.2 | 0.2 | 0.2 |
| Hydrogen | Corporation | | | | | | |
| Citrate | | | | | | | |
| MgSO₄•7H₂O | FUJIFILM Wako Pure Chemical | mg | 20 | 20 | 20 | 2.0 | 20 |
| | Corporation | | | | | | |
| MnSO₄•5H₂O | FUJIFILM Wako Pure Chemical | mg | 5 | 5 | 5 | 5 | 5 |
| | Corporation. | | | | | | |
| L-Cysteine- | Kanto Chemical Co., Inc. | mg | 50 | 50 | 50 | 50 | 50 |
| HCl | | | | | | | |
| 6′- | Biosynth Carbosynth | g | 0.2 | 0.2 | 0.2 | 0.2 | 0.2 |
| Sialyllactose | | | | | | | |
| (6′-SIA) | | | | | | | |
| β-Tocopherol | FUJIFILM Wako Pure Chemical | mg | 0 | 0.57 | 0.57 | 0.57 | 0.57 |
| | Corporation | | | | | | |
| γ-Tocopherol | FUJIFILM Wako Pure Chemical | mg | 0 | 14.1 | 18.9 | 22.5 | 25.2 |
| | Corporation | | | | | | |
| δ-Tocopherol | FUJIFILM Wako Pure Chemical | mg | 0 | 14.1 | 9.2 | 5.7 | 2.9 |
| | Corporation | | | | | | |
| γ/δ | | | | 1 | 2.1 | 3.9 | 8.7 |
| δ/β | | | | 24.6 | 16.1 | 10 | 5.1 |

TABLE 15
Table 4-4: Medium Composition

| | | | | Example 7-1 |
|---|---|---|---|---|
| | | | Comparative | Galactooligo- |
| | | | Example 7 | saccharide + |
| | | Unit/ | Galactooligo- | Tocopherol |
| | Reagent Source | 100 mL | saccharide | Pattern 1 |
| Peptone | Becton, Dickinson and Company | g | 1 | 1 |
| Beef extract | Becton, Dickinson and Company | g | 1 | 1 |
| Yeast extract | Becton, Dickinson and Company | g | 0.5 | 0.5 |
| Tween80 | nacalai tesque | g | 0.1 | 0.1 |
| K₂HPO₄ | Kokusan Chemical | g | 0.2 | 0.2 |
| Na Acetate | FUJIFILM Wako Pure Chemical | g | 0.5 | 0.5 |
| | Corporation | | | |
| Diammonium | Corporation | g | 0.2 | 0.2 |
| Hydrogen Citrate | FUJIFILM Wako Pure Chemical | | | |
| MgSO₄•7H₂O | FUJIFILM Wako Pure Chemical | mg | 20 | 20 |
| | Corporation | | | |
| MnSO₄•5H₂O | FUJIFILM Wako Pure Chemical | mg | 5 | 5 |
| | Corporation | | | |
| L-Cysteine-HCl | Kanto Chemical Co., Inc. | mg | 50 | 50 |
| Galactooligo- | FUJIFILM Wako Pure Chemical | g | 0.4 | 0.4 |
| saccharide | Corporation | | | |
| β-Tocopherol | FUJIFILM Wako Pure Chemical | mg | 0 | 0.57 |
| | Corporation | | | |
| γ-Tocopherol | FUJIFILM Wako Pure Chemical | mg | 0 | 14.1 |
| | Corporation | | | |
| δ-Tocopherol | FUJIFILM Wako Pure Chemical | mg | 0 | 14.1 |
| | Corporation | | | |
| α-Tocopherol | FUJIFILM Wako Pure Chemical | mg | — | — |
| | Corporation | | | |
| γ/δ | | | | 1 |
| δ/β | | | | 24.6 |

| | Example 7-2 | Example 7-3 | Example 7-4 | Example 7-5 |
|---|---|---|---|---|
| | Galactooligo- | Galactooligo- | Galactooligo- | Galactooligo- |
| | saccharide + | saccharide + | saccharide + | saccharide |
| | Tocopherol | Tocopherol | Tocopherol | α- |
| | Pattern 2 | Pattern 3 | Pattern 4 | Tocopherol |
| Peptone | 1 | 1 | 1 | 1 |
| Beef extract | 1 | 1 | 1 | 1 |
| WYeast extract | 0.5 | 0.5 | 0.5 | 0.5 |
| Tween80 | 0.1 | 0.1 | 0.1 | 0.1 |
| K₂HPO₄ | 0.2 | 0.2 | 0.2 | 0.2 |
| Na Acetate | 0.5 | 0.5 | 0.5 | 0.5 |
| Diammonium | 0.2 | 0.2 | 0.2 | 0.2 |
| Hydrogen Citrate | | | | |
| MgSO₄•7H₂O | 20 | 20 | 20 | 20 |
| MnSO₄•5H₂O | 5 | 5 | 5 | 5 |
| L-Cysteine-HCl | 50 | 50 | 5.0 | 50 |
| Galactooligo- | 0.4 | 0.4 | 0.4 | 0.4 |
| saccharide | | | | |
| β-Tocopherol | 0.57 | 0.57 | 0.57 | — |
| γ-Tocopherol | 18.9 | 22.5 | 25.2 | — |
| δ-Tocopherol | 9.2 | 5.7 | 2.9 | — |
| α-Tocopherol | — | — | — | 28.7 |
| γ/δ | 2.1 | 3.9 | 8.7 | |
| δ/β | 16.1 | 10 | 5.1 | |

### (2) Results

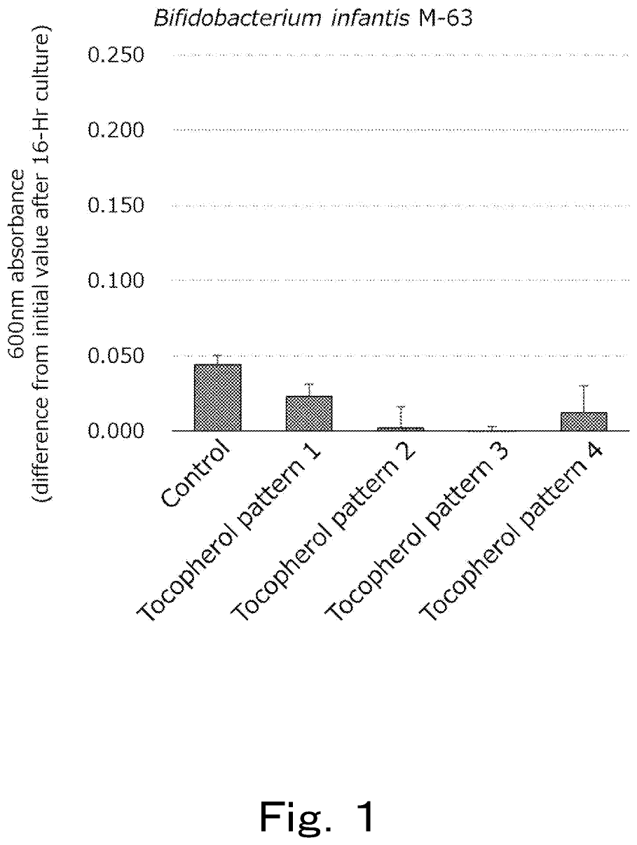

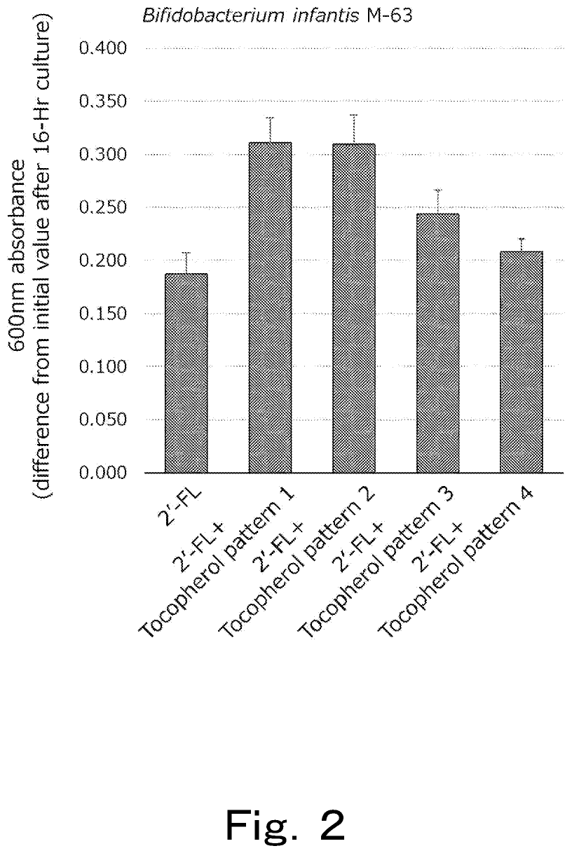

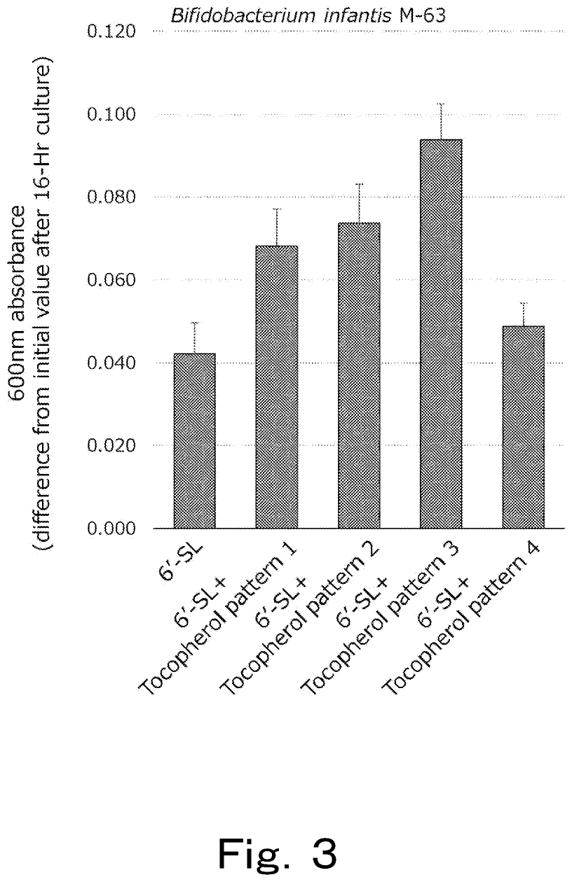

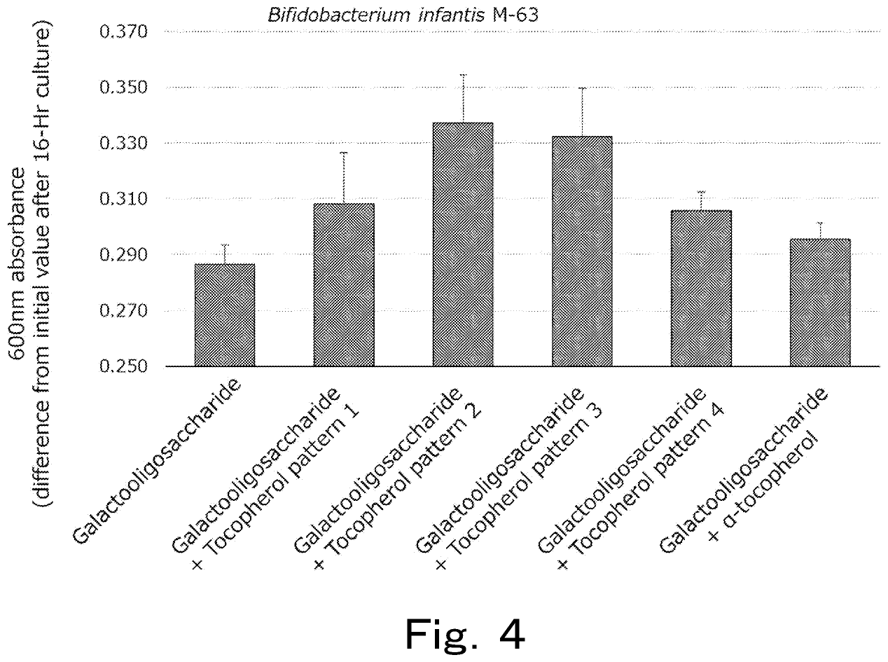

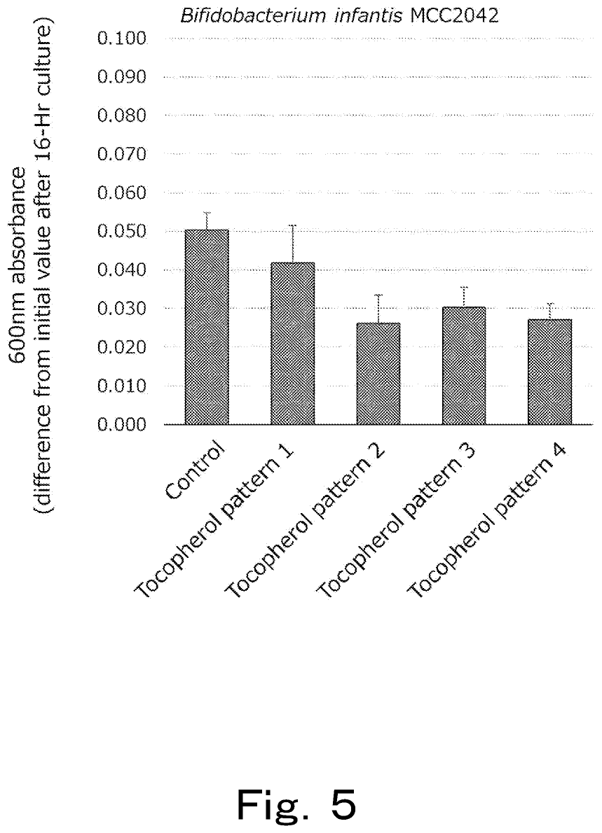

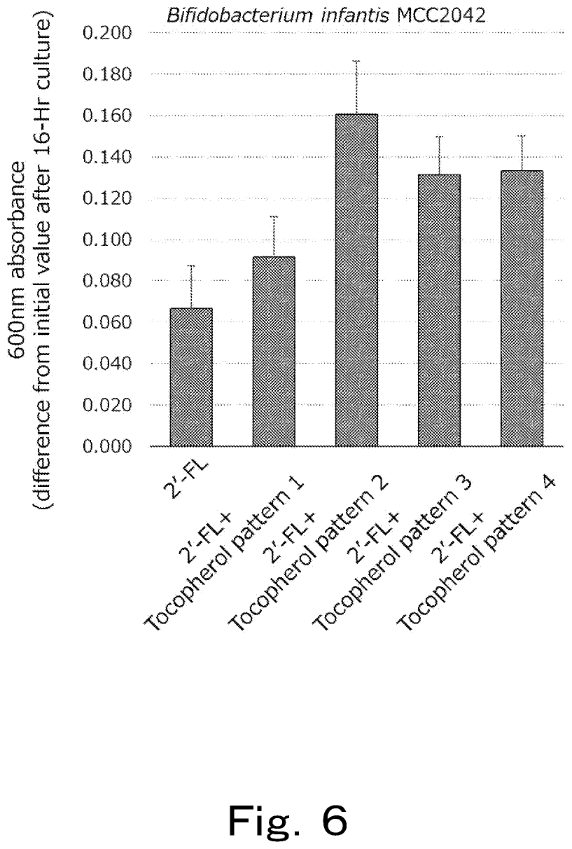

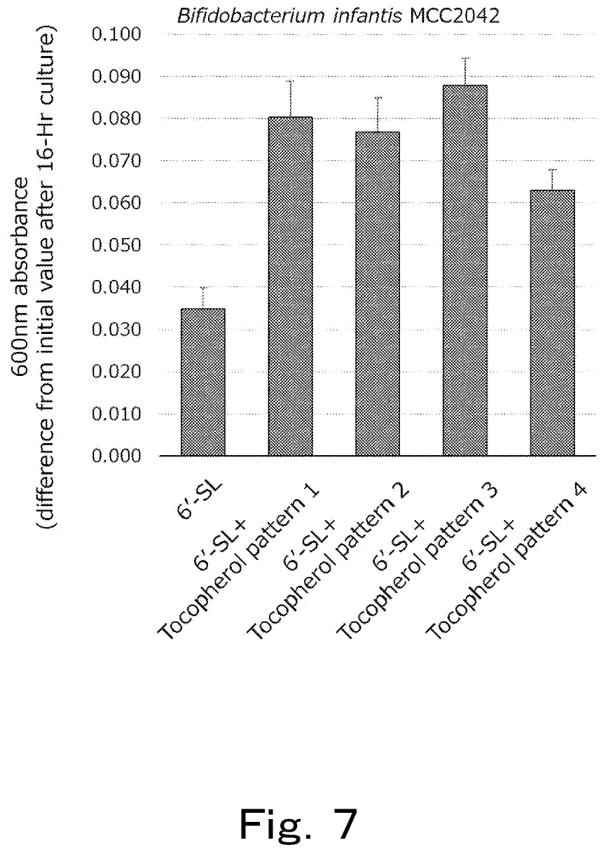

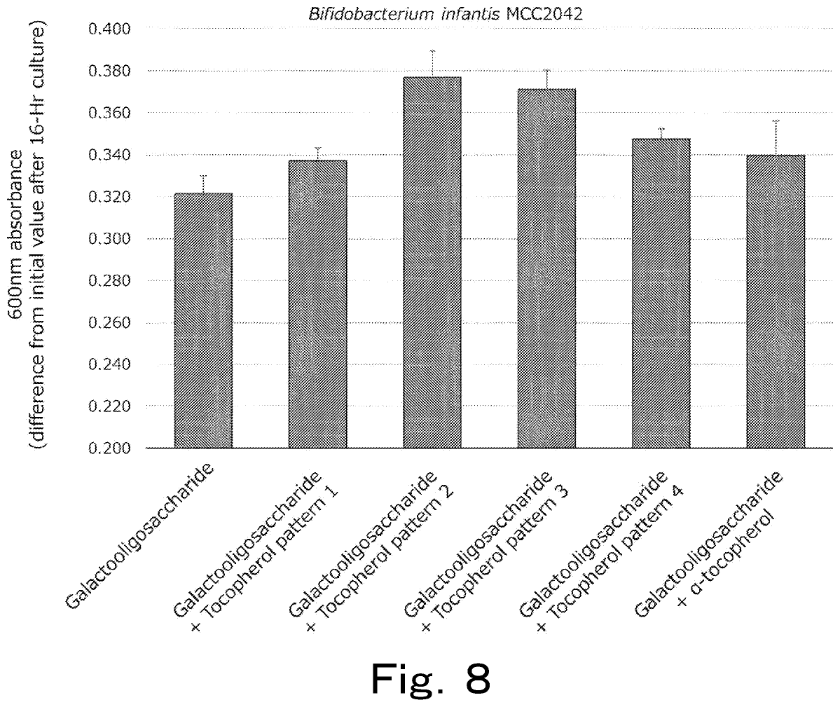

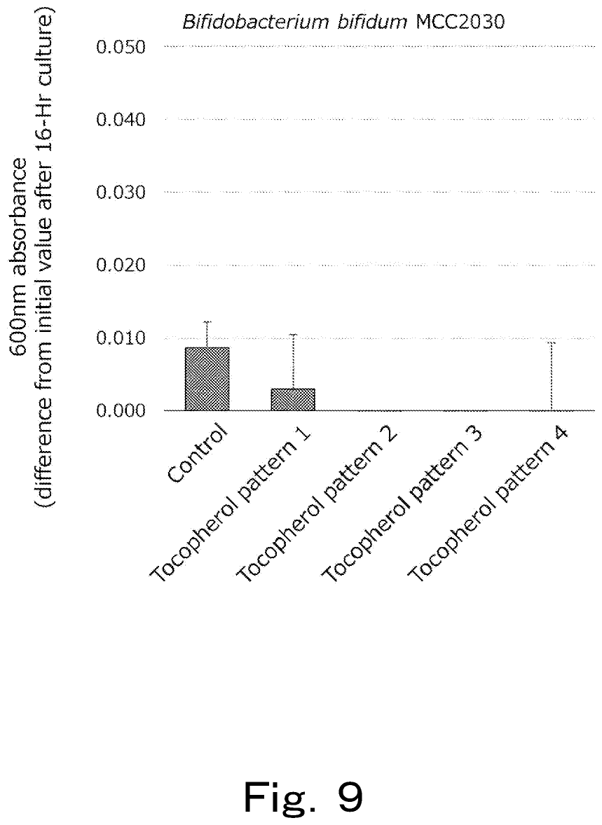

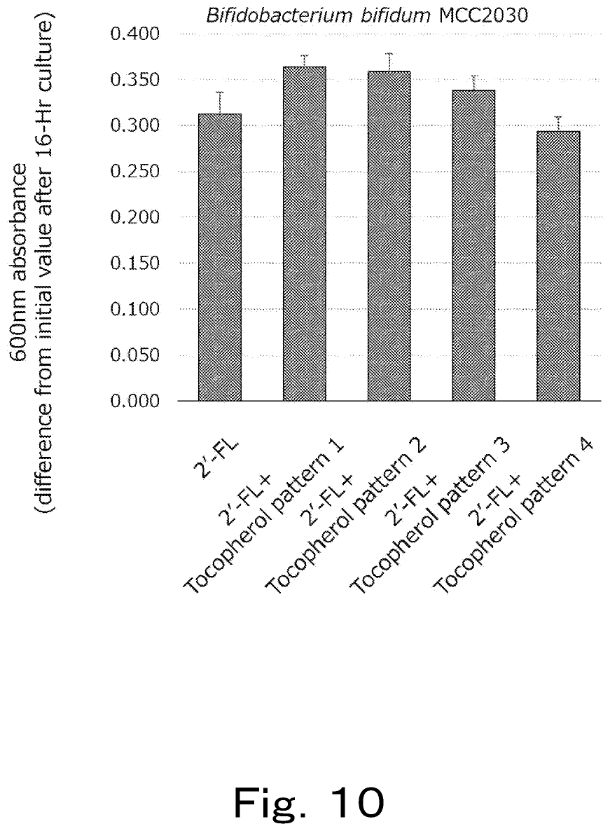

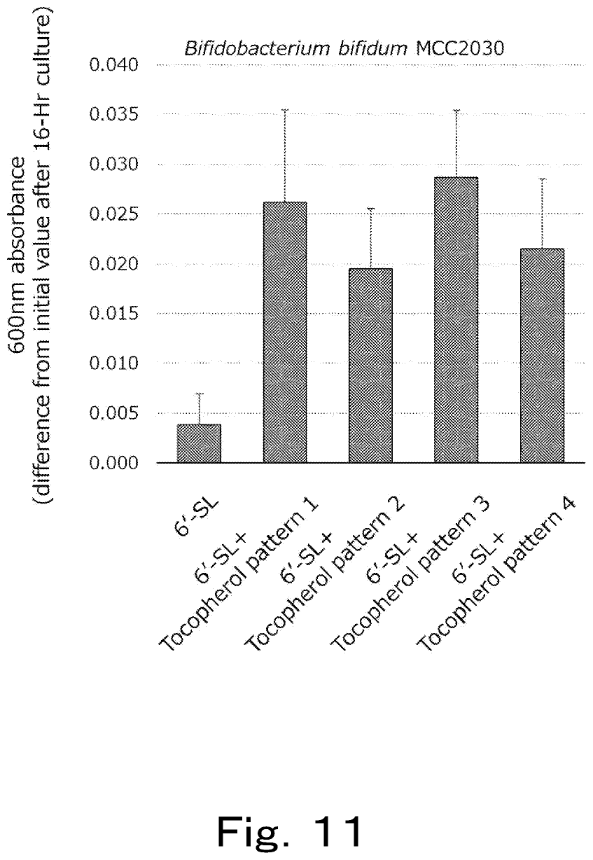

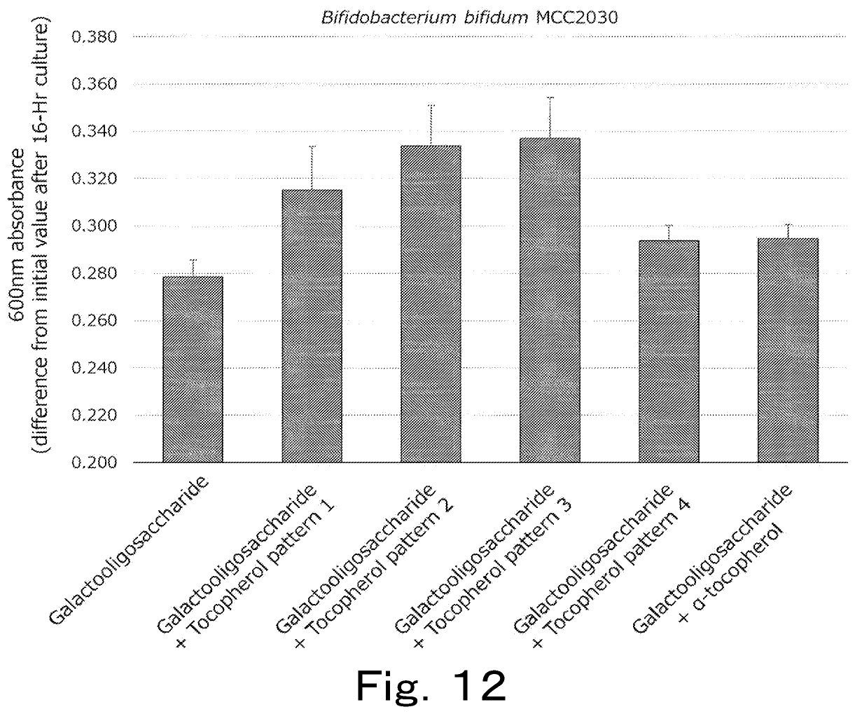

The differences in turbidities (ΔOD₆₀₀) between the beginning of the culture and after 16 hours of culture are shown in FIGS. 1 to 12 as the degrees of growth of the bifidobacterial strains. Regarding all the bacterial strains, the growth of the bifidobacterial strains was further promoted when the three kinds of tocopherol and an oligosaccharide were contained in combination compared to the cases containing an oligosaccharide but not containing the tocopherols. In the cases containing the three kinds of tocopherol but not containing the oligosaccharides, the growth of the bifidobacterial strains was not observed.

The GOS experiments showed a higher effect than α-tocopherol especially in patterns 2 and 3. In terms of experiments using 2′FL and 6′SL, there is no comparative data with α-tocopherol due to the rarity of HMOs. However, it would be plausibly anticipated that the result of experiments using 2′FL and 6′SL compared to α-tocopherol would be equal or greater than the result of the experiments using GOS. This is because GOS is included in human breast milk as well as 2′FL and 6′SL. Especially, the mixing rate on patterns 2 and 3 would be likely considered to show a higher effect than α-tocopherol as these rates show stably higher effect than α-tocopherol regardless of the differences on kinds of bifidobacterial strains.

### <Test Example 3> Evaluation of Bifidobacterial Strain Growth Promotion Action by Fecal Culture

### (1) Samples

The feces A and the feces B prepared in Test Example 1 described above were used as fecal samples.

### (2) Fecal Culture

To imitate the environment of the large intestine, a culture experiment was conducted by the following procedures. Bio Jr. 8 100 mLx8 culture apparatus (BJR-25NA1S-8M manufactured by Biott Corporation) was used, and the operations were according to the instructions of the device. As the medium, 100 mL of YCFA medium having the composition shown in Table 2 above was used. The medium components except for the tocopherols and the oligosaccharides were dissolved in purified water and sterilized in an autoclave, and the tocopherols and the oligosaccharides which were dissolved in dimethyl sulfoxide (DMSO, FUJIFILM Wako Pure Chemical Corporation) and in purified water, respectively, and which were then sterilized with a filter were added to the mixture after the autoclave treatment. The culture temperature was 37° C., and the anaerobic condition was maintained during the culture period by blowing filter-sterilized CO₂ into the culture vessels. After adjusting the pH to 7.0, 100 μL (10 mg in terms of feces) of the fecal samples, which were diluted with physiological saline, were added, and anaerobic culture was started. To imitate the pH in the large intestine of an infant, the pH was controlled by neutralizing with 1M Na₂CO₃ when the pH became lower than 5.5 during the culture period. The media were collected 7 hours after starting the culture and centrifuged at 8000 rpm for 3 min at 4° C., and the supernatants and the precipitates were collected.

### (3) DNA Extraction and Measurement of Bacterial Counts

Using GENE PREP STAR PI-480 automated isolation system (Kurabo Industries Ltd. Japan), DNA was extracted from the precipitates collected in (2). Using the extracted DNA as templates and using the primers of SEQ ID NO: 1 (ctcctggaaacgggtgg) and SEQ ID NO: 2 (ggtgttcttcccgatatctaca), quantitative PCR was conducted with Applied Bio systems 7500 Fast & 7500 Real-Time PCR System (manufactured by Thermo Fisher), and the bacterial counts of bifidobacterial strains were measured. The PCR conditions were 40 cycles of denaturation at 95° C. for 5 seconds, subsequent annealing at 55° C. for 20 seconds and elongation at 72° C. for 30 seconds.

### (4) Results

The bacterial counts of bifidobacterial strains after seven hours of culture, which were measured by the quantitative PCR, are shown in Table 5. The bacterial counts are the relative values where the bacterial counts of the groups without the addition of the tocopherols are regarded as 1.

The growth of bifidobacterial strains was further promoted with each of 2′-fucosyllactose, 6′-sialyllactose and galactooligosaccharide when used in combination with the tocopherols for the culture.

TABLE 16
Table 5-1

bifidobacterial Count
(Relative Value)

| | | |
|---|---|---|
| Group | Feces A | Feces B |

| | | | |
|---|---|---|---|
| Comparative | 2′-FL | 1 | 1 |
| Example 8: | | | |
| Example 8: | 2′-FL + Tocopherol | 1.8 | 2.6 |
| | Pattern 3 | | |

TABLE 17
Table 5-2

bifidobacterial Count
(Relative Value)

| | | |
|---|---|---|
| Group | Feces A | Feces B |

| | | | |
|---|---|---|---|
| Comparative | 6′-SL | 1 | 1 |
| Example 9: | | | |
| Example 9: | 6′-SL + Tocopherol | 1.9 | 3.9 |
| | Pattern 3 | | |

TABLE 18
Table 5-3

bifidobacterial Count
(Relative Value)

| | | |
|---|---|---|
| Group | Feces A | Feces B |

| | | | |
|---|---|---|---|
| Comparative | Control | 1 | 1 |
| Example 10-0: | | | |
| Comparative | Tocopherol | 0.42 | 0.32 |
| Example 10-1: | Pattern 1 | | |
| Comparative | Tocopherol | 0.26 | 0.32 |
| Example 10-2: | Pattern 4 | | |

TABLE 19
Table 5-4

| | | bifidobacterial Count | |
|---|---|---|---|
| | | (Relative Value) | |
| Group | | Feces A | Feces B |
| Comparative | Galactooligosaccharide | 1 | 1 |
| Example 11: | | | |
| Example 11-1: | Galactooligosaccharide + | 4.0 | 5.0 |
| | Tocopherol Pattern 1 | | |
| Example 11-2: | Galactooligosaccharide + | 8.5 | 6.0 |
| | Tocopherol Pattern 2 | | |
| Example 11-3: | Galactooligosaccharide + | 8.6 | 4.5 |
| | Tocopherol Pattern 3 | | |
| Example 11-4: | Galactooligosaccharide + | 8.1 | 4.8 |
| | Tocopherol Pattern 4 | | |

1.-14. (canceled)

15. A nutritional composition comprising an oligosaccharide and a tocopherol;
selected from the group consisting of β-tocopherol, γ-tocopherol, δ-tocopherol, and combinations thereof,
wherein the nutritional composition is not breast milk.

16. The nutritional composition according to claim 15, wherein the δ-tocopherol and the β-tocopherol have a mass ratio (δ/β) of 5 or more.

17. The nutritional composition according to claim 16, wherein the mass ratio (δ/β) is 10 or more.

18. The nutritional composition according to claim 15, wherein the γ-tocopherol the δ-tocopherol have a mass ratio (γ/δ) of 8.8 or less.

19. The nutritional composition according to claim 18, wherein the mass ratio (γ/δ) is 3.9 or less.

20. The nutritional composition according to claim 15, wherein the mass ratio of the total amount of β-tocopherol, γ-tocopherol, and δ-tocopherol to the total amount of oligosaccharide is 0.001 to 0.2.

21. The nutritional composition according to claim 15, wherein the total amount of β-tocopherol, γ-tocopherol, and δ-tocopherol relative to the entire composition is 0.0002 to 0.1 mass %.

22. The nutritional composition according to claim 15, wherein the amount of oligosaccharide in the composition is 0.05 to 10 mass %.

23. The nutritional composition according to claim 15, wherein the oligosaccharide has three or more, and five or less, sugar residues.

24. The nutritional composition according to claim 15, wherein the oligosaccharide comprises a human milk oligosaccharide.

25. The nutritional composition according to claim 24, wherein the human milk oligosaccharide is selected from the group consisting of lacto-N-neotetraose, lacto-N-tetraose, 2′-fucosyllactose, 3′-sialyllactose, 6′-sialyllactose, difucosyllactose, 3-fucosyllactose, and combinations thereof.

26. The nutritional composition according to claim 23, wherein the oligosaccharide comprises lacto-N-tetraose and 2′-fucosyllactose.

27. The nutritional composition according to claim 23, wherein the oligosaccharide comprises fructooligosaccharide and 6′-sialyllactose.

28. The nutritional composition according to claim 15, which is able to promote the growth of a bifidobacterial strain.

29. The nutritional composition according to claim 28, wherein the bifidobacterial strain is selected from the group consisting of Bifidobacterium breve, Bifidobacterium bifidum, Bifidobacterium infantis, Bifidobacterium longum, and combinations thereof.

30. The nutritional composition according to claim 15, which further contains a bifidobacterial strain.

31. The nutritional composition according to claim 15, which is a formula.
